(19) United States
(12) Patent Application Publication
(10) Pub. No.: US 2018/0228448 A1
(43) Pub. Date: Aug. 16, 2018

(54) INFORMATION PROCESSING SYSTEM, INFORMATION PROCESSING DEVICE, TERMINAL DEVICE, AND INFORMATION PROCESSING METHOD

(71) Applicant: SEIKO EPSON CORPORATION, Tokyo (JP)

(72) Inventors: Shigeyoshi MIYAZAWA, Chofu, Tokyo (JP); Hiroaki HOSOMI, Minowa-machi, Nagano (JP)

(21) Appl. No.: 15/746,709

(22) PCT Filed: Aug. 4, 2016
(86) PCT No.: PCT/JP2016/003610

§ 371 Date: Jan. 22, 2018

(30) Foreign Application Priority Data
Aug. 7, 2015 (JP) 2015-156784
Aug. 10, 2015 (JP) 2015-157973

(51) Int. Cl. A61B 5/00; A61B 5/11; G08B 25/10

(52) U.S. Cl. CPC A61B 5/747; A61B 5/1112; G08B 27/00; A61B 5/0022; G08B 25/10; A61B 5/681

(57) ABSTRACT

A guidance system acquires physiological information of a user by a wearable device; acquires positioning information identifying the position of the wearable device; and sends user information including physiological information and positioning information to an evacuation guidance server. The evacuation guidance server receives the user information sent by the wearable device, generates guidance service information based on the user information, and sends the guidance service information to the wearable device.

### CROSS REFERENCE TO RELATED APPLICATIONS

This is a U.S. National Phase application of International Patent Application No. PCT/JP2016/003610, filed on Aug. 4, 2016, which claims priority to Japanese Patent Application No. 2015-156784, filed on Aug. 7, 2015 and Japanese Patent Application No. 2015-157973, filed on Aug. 10, 2015. The entire disclosures of Japanese Patent Application Nos. 2015-156784 and 2015-157973 are hereby incorporated herein by reference.

### TECHNICAL FIELD

The present invention relates to an information processing system, information processing device, terminal device, and information processing method.

### BACKGROUND

Systems capable of determining the safety of a mobile terminal user and sending a message to a first responder as a result of mobile terminal user input to a mobile terminal possessed by the user during a disaster are known from the literature (see, for example, JP-A-2015-84167).

### SUMMARY

Because the system described in JP-A-2015-84167 cannot acquire bio-information of the user, information that is most appropriate to the individual user cannot be provided.

The system described in JP-A-2015-84167 also requires input by the mobile terminal user, and determines safety based on whether or not such input is received. However, a system that requires user input cannot easily acquire information quickly from a large number of users.

The invention is directed to the foregoing problem, and an objective of the invention is to provide an information processing system, information processing device, mobile terminal, and information processing method enabling acquiring bio-information about users, providing information appropriate to each user, and efficiently providing user support.

To achieve the foregoing objective, an information processing system according to the invention has a terminal device used by a user, and an information processing device capable of communicating with the terminal device; the terminal device including a physiological information acquisition unit that acquires physiological information of the user, a positioning information acquisition unit that acquires positioning information identifying the position of the terminal device, and a user information transmitter that sends user information including the physiological information acquired by the physiological information acquisition unit, and the positioning information acquired by the positioning information acquisition unit, to the information processing device; the information processing device including a user information receiver that receives the user information transmitted by the terminal device, a guidance service information generator that generates, based on the user information received by the user information receiver, guidance service information that is provided to the user, and a guidance service information transmitter that sends the guidance service information the guidance service information generator generated to the terminal device, and the terminal device including a display that displays specific grid coordinates converted from latitude and longitude.

Thus comprised, guidance service information generated based on the location of the terminal device and the physiological information of the user is sent to the terminal device of the user. As a result, guidance service information appropriate to the user can be provided, and multiple users can be quickly and efficiently supported. For example, when evacuation or rescue is required when a disaster occurs, guidance service information guiding users to evacuation sites, for example, can be provided.

The information processing system of the invention is further characterized by the guidance service information being information for guiding the user to a specific guidance location, and configured so that the guidance location is changed according to the physiological information of the user.

Thus comprised, users can be guided by the guidance service information to a guidance location, such as an evacuation center, when a disaster occurs. Because the guidance service information is configured to change the guidance location according to the physiological information of the user, the use can be guided appropriately to the physiological information of the user.

The information processing system of the invention is further characterized by the terminal device including a body contact unit that contacts the body of the user; the physiological information acquisition unit detecting bio-information through the body contact unit, and acquiring the detected bio-information as the physiological information; and the guidance service information generator of the information processing device generating the guidance service information based on the physiological information contained in the user information received by the user information receiver.

Thus comprised, bio-information of the user is sent from the terminal device to the information processing device, and guidance service information is generated based on the bio-information. As a result, guidance service information reflecting the physical condition of the user can be provided to the user. Furthermore, information related to the physical condition of the user of the terminal device can be collected by the information processing device, and information useful for determining the scale and condition of human suffering when a disaster occurs can be acquired.

To achieve the foregoing objective, another aspect of the invention is an information processing device capable of communicating with a terminal device used by a user, including: a user information receiver that receives, from the terminal device, user information including positioning information identifying the position of the terminal device, and physiological information of the user; a guidance service information generator that generates, based on the user information received by the user information receiver, guidance service information that is provided to the user; and a guidance service information transmitter that sends the guidance service information the guidance service information generator generated to the terminal device, the information processing device sending guidance service information including specific grid coordinates converted from latitude and longitude to the terminal device.

This configuration enables sending to the terminal device of the user guidance service information generated based on the location of the terminal device and the physiological information of the user. As a result, guidance service information appropriate to the user can be provided, and multiple users can be quickly and efficiently assisted. For example, when evacuation or rescue is required when a disaster occurs, guidance service information guiding users to evacuation sites, for example, can be provided.

The information processing device according to the invention is further characterized by having terminal-device-related information relating the terminal device to another terminal device; the guidance service information generator including, in the guidance service information corresponding to the terminal device, information related to another terminal device that is related to the terminal device by the terminal-device-related information.

This configuration can send to the user of the terminal device information related to users of other related terminal devices.

The information processing device according to the invention is further characterized by the guidance service information generator generating guidance service information corresponding to another terminal device based on the user information received by the user information receiver from the terminal device.

This configuration can send to the user of the terminal device guidance service information reflecting the condition of users of other related terminal devices.

The information processing device according to the invention is further characterized by the guidance service information generator generating the guidance service information to include positioning information in a format defined for the information processing device.

This configuration can include positioning information formatted for the information processing device in the guidance service information that is generated and then sent to the terminal device based on positioning information sent by the terminal device. As a result, positioning information in a different format than the positioning information the terminal device sends can be used by both the information processing device and terminal device to provide information related to guiding the user.

The information processing device according to the invention is further characterized by the guidance service information transmitter sending guidance service information sent to the terminal device to another predetermined destination.

This configuration can provide guidance service information provided to a user to a device used in the intended evacuation site, or a device used by an organization involved in search and rescue, and guidance service information can be used more effectively.

To achieve the foregoing objective, another aspect of the invention is a terminal device communicatively connected to an information processing device, including: a physiological information acquisition unit that acquires physiological information of the user of the terminal device; a positioning information acquisition unit that acquires positioning information identifying the position of the terminal device; a user information transmitter that sends user information including the physiological information acquired by the physiological information acquisition unit, and the positioning information acquired by the positioning information acquisition unit, to the information processing device; a guidance service information receiver that receives guidance service information transmitted from the information processing device corresponding to the user information sent by the user information transmitter; a body contact unit that contacts the body of the user; and a display that displays specific grid coordinates converted from latitude and longitude, the terminal device being configured to be usable when the body contact unit is touching the body of the user.

Thus comprised, guidance service information generated based on the location of the terminal device and the physiological information of the user can be sent to the terminal device of the user. As a result, guidance service information appropriate to the user can be provided, and multiple users can be quickly and efficiently supported. For example, when evacuation or rescue is required when a disaster occurs, guidance service information guiding users to evacuation sites, for example, can be provided.

The terminal device of the invention is further characterized by the physiological information acquisition unit detecting bio-information through the body contact unit, and acquiring the detected bio-information as the physiological information.

In this configuration, bio-information detected by contact with the body of the user is sent from the terminal device to the information processing device, and guidance service information is generated based on the bio-information. As a result, guidance service information reflecting the physical condition of the user can be provided to the user. Furthermore, when the information processing system is configured to combine terminal devices with an information processing device, information related to the physical condition of the user of the terminal device can be collected, and information useful for determining the scale and condition of human suffering when a disaster occurs, for example, can be acquired.

The terminal device of the invention is further characterized by having terminal storage that stores physical characteristics related to the body of the user; the physiological information acquisition unit acquiring the physiological information based on the physical characteristics stored by the terminal storage.

By previously storing physical characteristics related to the user in the terminal device, this configuration can provide to the user guidance service information reflecting those physical characteristics. For example, if the physical characteristics include information related to medical treatment, guidance service information appropriate to the medical needs of individual users can be provided to the users.

The terminal device of the invention is further characterized by the positioning information acquisition unit detecting the position of the terminal device and generating positioning information identifying the detected position.

This configuration can provide more appropriate guidance service information to users because positioning information indicating the most recent location of the terminal device can be sent to the information processing device.

The terminal device of the invention is further characterized by having a terminal display that displays guidance service information received by the guidance service information receiver; the terminal display displaying positioning information contained in the guidance service information as positioning information in a format defined for the information processing device.

This configuration enables using positioning information of a format different from the positioning information the terminal device sends to provide evacuation information to the user. Therefore, positioning information suitable for providing guidance to a specific location can be used without being limited by the specifications of the positioning information the terminal device detects.

The terminal device of the invention is further characterized by a power generator that produces power, and a power supply unit that supplies at least power generated by the power generator to the user information transmitter and guidance service information receiver.

This configuration avoids the terminal device shutting down due to insufficient power, and increases the likelihood of being able to use the terminal device in a disaster.

The terminal device of the invention is further characterized by a motion detector that detects movement of the terminal device; the user information transmitter sending the detection result of the motion detector to the information processing device.

This configuration can indirectly detect conditions related to the user possessing the terminal device based on movement of the terminal device, and provide to the user guidance service information reflecting the condition of the user.

To achieve the foregoing objective, another aspect of the invention is an information processing method using a terminal device used by a user, and an information processing device capable of communicating with the terminal device, including steps of: the terminal device acquiring physiological information of the user, acquiring positioning information identifying the position of the terminal device, and sending user information including the physiological information and the positioning information to the information processing device; and the information processing device receiving the user information transmitted by the terminal device, generating guidance service information that is provided to the user including specific grid coordinates converted from latitude and longitude based on the user information, and sending the guidance service information to the terminal device.

Thus comprised, guidance service information generated based on the location of the terminal device and the physiological information of the user is sent to the terminal device of the user. As a result, guidance service information appropriate to the user can be provided, and multiple users can be quickly and efficiently supported. For example, when evacuation or rescue is required when a disaster occurs, guidance service information guiding users to evacuation sites, for example, can be provided.

Another information processing system according to the invention is characterized by including a terminal device used by a user, and an information processing device capable of communicating with the terminal device; the terminal device including a physiological information acquisition unit that acquires physiological information of the user, a positioning information acquisition unit that acquires positioning information identifying the position of the terminal device, a terminal receiver that receives a query sent by the information processing device, and a user information transmitter that, when the terminal receiver receives the query, sends user information including the physiological information acquired by the physiological information acquisition unit, and the positioning information acquired by the positioning information acquisition unit, to the information processing device; the information processing device including a query transmitter that sends a query to the terminal device, and a user information receiver that receives the user information from the terminal device, and the terminal device including a display that displays specific grid coordinates converted from latitude and longitude.

This configuration enables the information processing device to control transmission of information from the terminal device of a user. As a result, because information related to the condition of the users of the terminal devices can be collected by the information processing device, information can be quickly and efficiently collected from many users.

The information processing system according to the invention is further characterized by the information processing device receiving the user information from multiple terminal devices, and displaying disaster conditions based on the user information.

Using information the information processing device collects from terminal devices, this configuration enables displaying conditions based on the positioning information and physiological information of the users. As a result, the condition of users using the terminal devices can be visually understood.

The foregoing information processing device may be configured to generate user condition information correlating and mapping the locations of multiple terminal devices and physiological information contained in the user information, and display the information, based on user information received from multiple terminal devices.

This configuration enables visually understanding the locations and conditions of multiple users using the terminal devices.

The information processing system according to the invention is further characterized by the information processing device having user information storage that stores information combining the positioning information and the physiological information contained in the user information received by the user information receiver.

By storing information received from user terminal devices, this configuration can easily use the collected information. By interpreting or tabulating information received from the user terminal devices, the scale and condition of damage can be determined in detail when a disaster occurs, for example.

The information processing system of the invention is further characterized by the physiological information acquisition unit of the terminal device including a bio-information detector that contacts the body of the user and detects bio-information, and acquires, and transmits by the user information transmitter, the bio-information detected by the bio-information detector as the physiological information.

As a result, extremely timely, more detailed information related to the physical status of the user of the terminal device can be collected.

The information processing system of the invention is further characterized by the terminal device being connected to the information processing device through a communication network including communication devices disposed appropriately in a region; and the query transmitter of the information processing device specifying a region for the communication devices when sending the query to the terminal devices.

This configuration enable specifying the area from which terminal devices are controlled to send information. As a result, information related to the status of users 7 using terminal devices in a designated area can be collected by the information processing device.

The information processing system of the invention is further characterized by the physiological information acquisition unit of the terminal device including a bio-information detector that contacts the body of the user and detects bio-information, and acquiring the bio-information detected by the bio-information detector as the physiological information; and the guidance service information generator of the information processing device generating the guidance service information based on the physiological information in the user information the user information receiver receives.

This configuration enables the information processing device to control transmission of information from the terminal device of a user. As a result, because information related to the condition of the users of the terminal devices can be collected by the information processing device, information can be quickly and efficiently collected from many users.

To achieve the foregoing objective, the invention is further characterized by an information processing device capable of communicating with a terminal device used by a user, including: a transmitter that sends a query to the terminal device; and a user information receiver that receives user information that is sent by the terminal device in response to a query sent by the transmitter, and includes positioning information identifying the position of the terminal device, and physiological information of the user, the information processing device sending guidance service information including specific grid coordinates converted from latitude and longitude to the terminal device.

This configuration enables the information processing device to control transmission of information from the terminal device of a user. As a result, because information related to the condition of the users of the terminal devices can be collected by the information processing device, information can be quickly and efficiently collected from many users.

To achieve the foregoing objective, another aspect of the invention is an information processing method using a terminal device used by a user, and an information processing device capable of communicating with the terminal device, and including: the terminal device acquiring physiological information of the user, acquiring positioning information identifying the position of the terminal device, receiving a query transmitted by the information processing device, and when the query is received, sending user information including the physiological information and specific grid coordinates converted from latitude and longitude to the information processing device; and the information processing device sending a query to the terminal device, and receiving the user information from the terminal device.

This configuration enables the information processing device to control transmission of information from the terminal device of a user. As a result, because information related to the condition of the users of the terminal devices can be collected by the information processing device, information can be quickly and efficiently collected from many users.

To achieve the foregoing objective, a terminal device of the invention is characterized by being used in the foregoing information processing system.

This configuration enables an information processing device that communicates with terminal devices to collect information from the terminal device a user uses.

When the foregoing information processing device and terminal device are configured with a computer the foregoing embodiments can also be configured by a program enabling the functions, or a computer-readable recording medium storing the program. The information processing methods can also be configured as a program enabling the information processing device and terminal device to execute the information processing method, and the program can be stored on a computer-readable recording medium. Examples of such computer-readable recording media include floppy disk, HDD (Hard Disk Drive), CD-ROM (Compact Disk Read Only Memory), DVD (Digital Versatile Disk), Blu-ray® Disc, magneto-optical disc, nonvolatile memory cards, internal storage devices of an image display device (RAM (Random Access Memory), ROM (Read Only Memory), other semiconductor memory), and external storage devices (such as USB (Universal Serial Bus) memory devices).

### BRIEF DESCRIPTION OF DRAWINGS

FIG. 1 illustrates a guidance system according to a first embodiment of the invention.

FIG. 2A is a function block diagram of a wearable device of the guidance system.

FIG. 2B is a function block diagram of a gateway device of the guidance system.

FIG. 3 is a function block diagram of an evacuation guidance server of the guidance system.

FIG. 4 shows an example of a UTM grid map.

FIG. 5A illustrates the relationship between UTM grid coordinates and latitude and longitude.

FIG. 5B illustrates the format of UTM grid coordinates.

FIG. 6 is a flow chart of the operation of the guidance system.

FIG. 7 is a flowchart of the operation of the evacuation guidance server.

FIG. 8A shows an example of guidance service information displayed on a wearable device.

FIG. 8B shows an example of guidance service information displayed on a wearable device.

FIG. 9 is a flow chart of the operation of a guidance system according to a second embodiment of the invention.

FIG. 10 is a flow chart showing the operation of the guidance system.

FIG. 11 shows an example of a disaster map displayed by the evacuation guidance server.

### DESCRIPTION OF EMBODIMENTS

Embodiments of the invention are described below with reference to the figures.

### Embodiment 1

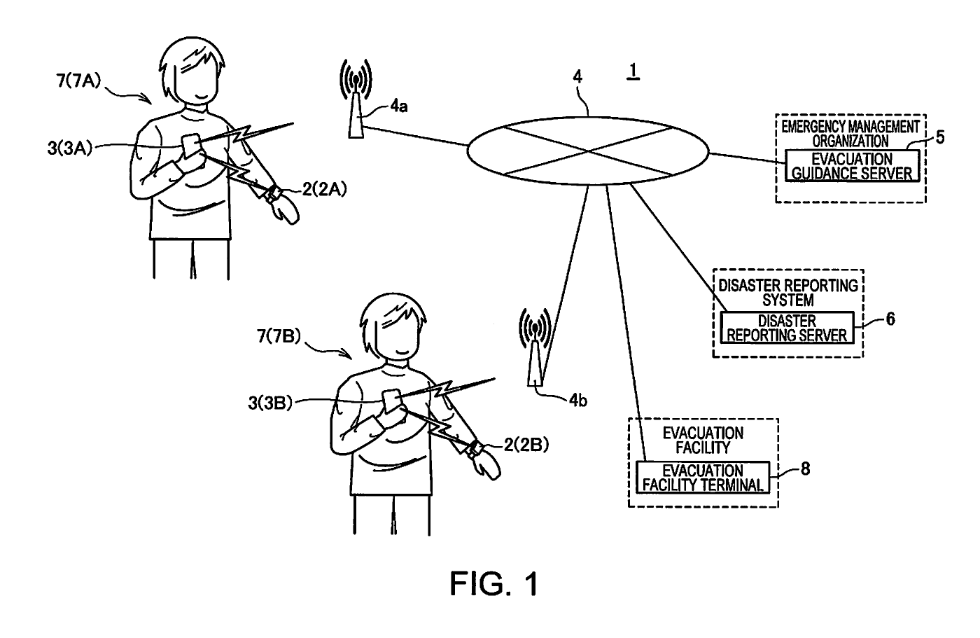

FIG. 1 schematically illustrates the configuration of a guidance system 1 (information processing system) according to a first embodiment of the invention. The guidance system 1 includes a wearable device 2 (terminal device) worn by a user 7, and an evacuation guidance server 5 (information processing device) that communicatively connects to the wearable device 2. The wearable device 2 and evacuation guidance server 5 can communicate with each other, and the guidance system 1 is a system that provides users with information useful for guidance to evacuation centers by means of the evacuation guidance server 5.

The wearable device 2 is a terminal device that the user 7 holds or carries, or wears attached to the user's body or clothing, and is a relatively small device. The guidance system 1 supposes that one wearable device 2 is used by one user 7.

As described below, the wearable device 2 is a mobile information processing device having at least a positioning function for acquiring the current location, and a communication function for communicating by near-field communication with a gateway device 3. In this embodiment, the wearable device 2 is a wristwatch-type device that can be worn on the wrist by the user 7, and is an example of currently popular IoT (Internet of Things) devices.

Note that the wearable device 2 may take various forms as a mobile information processing device that can be worn by the user 7, including eyeglasses, rings, and clothing.

The wearable device 2 also has a function for acquiring information related to the body, and more specifically physiological information related to the medical or physiological status, of the user 7. The physiological information is information that affects selection of the evacuation site when the user 7 is guided to an evacuation site, and/or selecting the evacuation route from the current location of the user 7 to the evacuation site.

In this embodiment of the invention the physiological information includes information acquired by measurement or detection by the wearable device 2. For example, the physiological information may include vital information acquired by a measurement or detection process applied by the wearable device 2 to the user's body. Vital information includes information acquired by directly or indirectly measuring or detecting information about the body of the user 7, any may also be referred to as bio-information or medical information related to the user's body. More specifically, vital information as used here may be narrowly defined vital signs (pulse rate, blood pressure, blood oxygen saturation, and body temperature), or more broadly defined vital signs including brain waves, body fat percentage, and blood type.

Physiological information may also include a standard value or reference value statistically acquired from vital information the wearable device 2 measured or detected in the past. The physiological information is also not limited to information acquired by measurement or detection of the body of the user 7, and may include, for example, the speed of travel of the user calculated from location information.

In addition to information measured or detected by the wearable device 2, the physiological information acquired by the wearable device 2 may include physical characteristics that are previously set or input. Such physical characteristics are information related to the individual user 7, and is not specifically limited as long as the information pertains to the body (organism), health, or medical care of the user 7. For example, information pertaining to the body (organism) of the user includes the age, sex, height, weight body fat percentage, and blood type of the user. The physical characteristics may also include information related to the disease (illness) of the user, or a disease being treated. Treatment information related to the treatment for a current or past disease may also be included. The physical characteristics may also include information related a user's disability or past medical history. Treatment information includes, for example, information related to care the user requires continuously (such as dialysis), or information related to the user's medication (drug) history. Treatment information may also include information related to internal medical devices such as a pacemaker, artificial joints, stoma, screws and plates used for orthopedic treatment, clips, stents, and artificial lenses.

The number of users 7 that can use the guidance system 1 is not specifically limited, and multiple users 7 may use the guidance system 1. In this example, two users 7 (users 7A and 7B) wear wearable devices 2A and 2B, respectively. In this example, user 7A and user 7B are collectively referred to as user 7, and wearable device 2A and wearable device 2B are collectively referred to as wearable device 2.

Users 7A, 7B also have a gateway device 3A, 3B, respectively. The gateway devices 3A, 3B are, as described below, mobile devices such as a smartphone or wireless router having a wireless communication capability gateway devices 3A, 3B are collectively referred to as gateway device 3.

The wearable device 2 and evacuation guidance server 5 communicate with each other through a wireless network 4. The wireless network 4 comprises wide-area communication lines such as a public telephone network or a dedicated line, and may include an open network such as the Internet, or part or all of the network may be a closed network. The wireless network 4 may include network equipment not shown, such as gateway devices, server devices, and routers managed by a company or group such as a telephone company or cell phone company (communication carrier).

In this embodiment of the invention, the evacuation guidance server 5 is connected to the wireless network 4 through such communication devices.

Furthermore, by communicating wirelessly with relay devices 4a, 4b connected to the wireless network 4, the wearable device 2 connects through the wireless network 4 to the evacuation guidance server 5. The wearable device 2 may communicate directly wirelessly with the relay devices 4a, 4b, but in this embodiment of the invention communicates with the relay devices 4a, 4b through a gateway device 3.

A gateway device 3 is a device that relays communication between the wearable device 2 and devices connected to the wireless network 4 (including an evacuation guidance server 5 and a disaster reporting server 6). The gateway device 3 has at least communication functions for communicating by near-field communication with the wearable device 2 and communicating with devices connected to the wireless network 4, and a positioning function for determining its current location.

FIG. 1 illustrates an example in which a smartphone (mobile communication terminal) carried by the user 7 and able to access the wireless network 4 through a mobile communication network is used as the gateway device 3.

The wearable device 2A and gateway device 3A carried by user 7A communicate by near-field communication. The gateway device 3A functions as a relay device that provides, to the wearable device 2A, a wireless communication connection between the relay device 4a and gateway device 3A. By a function of the gateway device 3A, the wearable device 2A exchanges data with a device, such as the evacuation guidance server 5, connected to the wireless network 4. Wearable device 2B is likewise configured, that is, gateway device 3B connected to the relay device 4b operates as a relay device for the wearable device 2B.

Some possible configurations of the gateway device 3 are described below. The gateway device 3 may be a mobile communication device other than a smartphone, such as a simple cell phone or wireless router. The gateway device 3 may also be configured as a moving communication terminal (such as an airship, drone, or other aircraft, or a vehicle that travels on roads), or a stationary communication terminal affixed to the ground or a building. The moving or stationary communication terminal may be a base station forming part of a wireless communication network. For example, a moving base station may be configured as an airship or drone (unmanned aerial vehicle) that is used during a disaster to restore a mobile communication network in disaster areas and is capable of deploying a wireless LAN or other near-field communication zone as needed. A stationary base station may be installed on each floor of a building, for example, and capable of creating a wireless LAN or other near-field communication area as needed.

When the wearable device 2 has a communication function for communicating wirelessly with the relay devices 4a, 4b, wearable device 2A may communicate directly with relay device 4a, and wearable device 2B may communicate directly with relay device 4b. In this case, the wearable device 2 does not use the relay capability of the gateway device 3, communicates through the wireless network 4 with the evacuation guidance server 5, and the user 7 can use functions of this embodiment of the invention without having the gateway device 3.

In this embodiment, a near-field communication unit 15 embodies a user information transmitter and a guidance service information receiver. When the gateway device 3 relays wearable device 2 communications, the near-field communication unit 15 and gateway device 3 (particularly the communicator 22 and near-field communication unit 25 of the gateway device 3) work together as the user information transmitter and guidance service information receiver.

The guidance system 1 includes a evacuation guidance server 5, disaster reporting server 6, and evacuation facility terminal 8 communicatively connected to the wireless network 4.

The evacuation guidance server 5 is a server managed by an emergency management organization that develops and implements disaster response plans when a disaster occurs, and is a server of a service provider that provides information useful for evacuation guidance to multiple users including users 7 through communication networks including the wireless network 4.

Disasters to which the guidance system 1 is directed are emergencies creating a need to take refuge, and are not limited to natural disasters such as earthquakes, wind and water damage, and volcanic eruptions, and include disasters from war and terrorism, fire, and accidents. The guidance system is also not limited to addressing all cited disasters, and may be directed to one or more particular types of emergencies.

The guidance system 1 provides guidance service information to users 7 by the evacuation guidance server 5. Guidance service information is information informing the user 7, and may include, for example, information identifying the place or location to which directions are provided, and information related to the route to the destination evacuation site, navigational directions, and modes of travel. The guidance service information can be information for enabling the user 7 to take refuge, or instructing the user 7 to take refuge. In this case, the guidance service information includes where to take refuge and information describing the evacuation route to the refuge location. The guidance service information the evacuation guidance server 5 provides may be common information provided to all users 7, but in this embodiment the evacuation guidance server 5 generates guidance service information specific to the situation of each user 7A, 7B. The guidance service information can obviously also be information to guide the user 7 for purposes other than evacuation.

The disaster reporting server 6 is an announcement server in a disaster reporting system that reports disasters, and reports a disaster through a communication network including the wireless network 4. For example, the disaster reporting server 6 may broadcast earthquake reports and tsunami warnings broadcast by a national meteorological agency, and emergency and evacuation information broadcast by national and regional government authorities. The evacuation guidance server 5 is also not specifically limited to distributing information from these sources, and in this example distributes information from at least the disaster reporting server 6. The evacuation facility terminal 8 is a terminal device used in a facility (refuge site) that can be used as the destination for user 7 evacuation, and is operated, for example, by an employee of the evacuation facility.

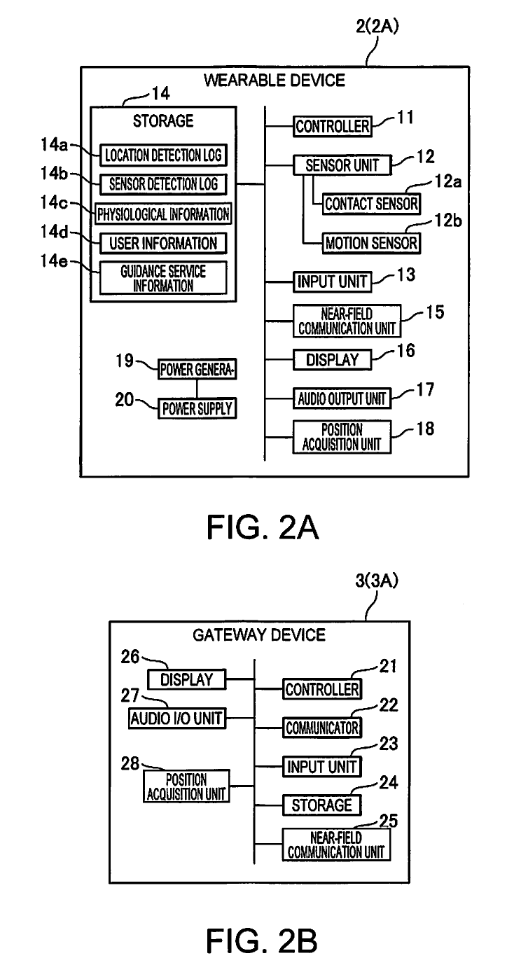

FIG. 2A is a function block diagram of a wearable device 2 in the guidance system 1, and FIG. 2B is a function block diagram of a gateway device 3 in the guidance system 1. Wearable devices 2A and 2B are configured identically, and gateway devices 3A, 3B are configured identically.

The wearable device 2 has a controller 11, sensor unit 12, input unit 13, storage 14, near-field communication unit 15, display 16, audio output unit 17, and position acquisition unit 18.

The controller 11 has the configuration of a computer including a CPU, ROM, and RAM. The controller 11 functions as an information processor that centrally controls other parts and operating processes by executing a basic control program stored in ROM, and other programs stored in storage 14.

The sensor unit 12 (physiological information acquisition unit) includes a contact sensor 12a and motion sensor 12b. The contact sensor 12a (body contact unit) has sensors for acquiring vital information about the user 7, the wearer. In this example, the contact sensor 12a contacts the body of the user 7 and detects or measures vital information. More specifically, the contact sensor 12a contacts the skin of the user 7, and detects vital signs (pulse rate, blood pressure, blood oxygen saturation, body temperature). By emitting light to the skin of the user 7, and measuring light absorption or reflection, the contact sensor 12a detects the user's pulse rate and blood oxygen saturation, for example. In the simplest example, the contact sensor 12a detects the pulse rate of the user 7. The contact sensor 12a may also have a temperature sensor and detect the body temperature of the user 7. Further alternatively, the contact sensor 12a may have a brain wave sensor for detecting the brain waves of the user 7. The contact sensor 12a may thus be configured with a light sensor and temperature sensor, or as a sensor unit with multiple sensors.

The motion sensor 12b (motion detector) is a sensor that detects movement of the wearable device 2, and may be, for example, an acceleration sensor (including a gravity sensor) that detects acceleration, or angular velocity sensor that detects angular velocity. The motion sensor 12b may also be a geomagnetic field sensor (also called an orientation sensor). The motion sensor 12b may also be configured as an inertial sensor unit integrating two or more of an acceleration sensor, angular velocity sensor, and geomagnetic field sensor. The motion sensor 12b may also include a barometric pressure sensor (also called an altitude sensor).

The input unit 13 comprises operating switches not shown and a touch panel, for example, disposed to the display 16, and inputs user 7 instructions by detecting user 7 operations.

The storage 14 nonvolatilely stores control programs and data required by the wearable device 2 to execute processes.

The near-field communication unit 15 is a device that communicates wirelessly by near-field communication with the gateway device 3, for example, as controlled by the controller 11, and in this embodiment communicates by Bluetooth (R), wireless LAN, or ZigBee®.

The display 16 (terminal display unit) has an LCD panel, OLED (Electro-Luminescence) panel, e-paper, or other type of display panel, and displays information for the user 7 as controlled by the controller 11. The audio output unit 17 includes an amplifier and speaker, and outputs audio to the user 7 as controlled by the controller 11.

The position acquisition unit 18 (positioning information acquisition unit) acquires positioning information indicating the current location of the wearable device 2. For example, the position acquisition unit 18 may use GPS (Global Positioning System) to detect the current position and generate positioning information. In this case, the position acquisition unit 18 receives radio signals through a GPS antenna not shown from GPS satellites, and calculates the current position from latitude and longitude. Note that the position acquisition unit 18 may be comprised without a configuration for location detection. For example, the position acquisition unit 18 may acquire by the near-field communication unit 15 the current location of the gateway device 3 that the gateway device 3 calculates. Because the wearable device 2 and gateway device 3 are terminals possessed by the user 7, and are located within the communication range of the near-field communication unit 15, the location detected by the gateway device 3 can be used as the location of the wearable device 2.

The storage 14 stores a location detection log 14a, sensor detection log 14b, physiological information 14c, user information 14d, and guidance service information 14e.

The location detection log 14a is a log of locations detected or acquired by the position acquisition unit 18. The location detection log 14a may record information indicating the position acquired by the position acquisition unit 18, and may relationally store the acquired position to the date and time the position was acquired. The location detection log 14a may also record the results of the controller 11 statistically processing the locations acquired by the position acquisition unit 18.

The sensor detection log 14b is a log of the results (referred to below as detection results) detected or measured by the contact sensor 12a and motion sensor 12b of the sensor unit 12. The sensor detection log 14b may record simply the detection results, or information relating a detection result to the date and time the result was detected. The sensor detection log 14b can use change in the vital information of the user 7, or information related to user 7 movement (such as acceleration, angular velocity, direction of travel (orientation), change in altitude (pressure change)).

Based on the detection results of the contact sensor 12a and motion sensor 12b, the controller 11 may evaluate the medical or physiological status of the user 7, and include the determined result stored in the sensor detection log 14b in the storage 14. For example, the controller 11 may evaluate the health status of the user 7 based on the detection results from the contact sensor 12a, and/or past detection results contained in the sensor detection log 14b. The controller 11 may also calculate an index related to the detection results of the contact sensor 12a based on the detection results from the contact sensor 12a, and/or past detection results contained in the sensor detection log 14b. For example, the controller 11 may calculate an index to the normal heart rate or the blood pressure range of the user 7.

Based on the location detection log 14a, the controller 11 may also calculate the amount of activity of the user 7, and include the resulting amount of activity in the sensor detection log 14b stored in the storage 14.

Note that the controller 11 may use methods known from the literature in the process calculating the amount of activity and other processes.

The physiological information 14c and user information 14d are information the controller 11 generates to send to the evacuation guidance server 5. The controller 11 generates the physiological information 14c based on the sensor detection log 14b. The user information 14d is information the controller 11 generates based on the physiological information 14c and location detection log 14a. In other words, the user information 14d includes the location detection log 14a and physiological information 14c, and the physiological information 14c is generated from the sensor detection log 14b. The information the physiological information 14c and user information 14d contain may be all of the information, or only part of the information, in the location detection log 14a and sensor detection log 14b; or information generated by processing the location detection log 14a and sensor detection log 14b. The specific format of the physiological information 14c and user information 14d the controller 11 generates may be previously set in the wearable device 2, or specified by data the evacuation guidance server 5 sends to the wearable device 2. The user information 14d may include the transportation means (by foot, bicycle, or automobile, for example) the user 7 uses in an emergency, or information related to a wheelchair, cane, or other assistive device used in an emergency.

The user information 14d may include physical characteristics as described above, in which case the storage 14 functions as a terminal storage unit. Physical characteristics are previously input or set, and stored in the storage 14. The physical characteristics may obviously be stored in the storage 14 as information separate from the user information 14d.

The guidance service information 14e is information the wearable device 2 receives from the evacuation guidance server 5. When guidance service information the evacuation guidance server 5 transmits is received through the gateway device 3, the controller 11 first stores the guidance service information as guidance service information 14e in the storage 14. Based on the guidance service information 14e, the controller 11 displays text and maps indicating where to take refuge and the name of the facility, the location of the evacuation site, and the route to the evacuation site, on the display 16.

The wearable device 2 also has a power generator 19, and outputs power generated by the power generator 19 to the power supply 20. The power generator 19 may use a wide range of known devices and methods, including solar power generation, thermoelectric generation, and devices that use the kinetic energy of a rotary pendulum that turns with movement of the user's 7 wrist. The power supply 20 (power supply unit) supplies operating power to other parts of the wearable device 2. The power supply 20 may comprise a storage battery or capacitor that stores power generated by the power generator 19. By using a power generator 19, the wearable device 2 can continue operating without losing power in times of emergency.

As shown in FIG. 2B, the gateway device 3 includes a controller 21, communicator 22, input unit 23, storage 24, near-field communication unit 25, display 26, audio I/O unit 27, and position acquisition unit 28.

The controller 21 includes a CPU, ROM, and RAM. The controller 21 functions as an information processor that centrally controls other parts and operating processes by executing a basic control program stored in ROM, and other programs stored in storage 24.

The communicator 22 communicates as controlled by the controller 21 according to a specific wireless communication network standard, for example. As a result, the gateway device 3 can use services (such as disaster reports from a telephone or cell phone company) that use a wireless communication network, and can use services from the wireless network 4 through the wireless communication network. The communicator 22, as controlled by the controller 21, relays communication between the wearable device 2 and wireless network 4. As a result, the wearable device 2 can access the wireless network 4, and receive data from servers connected to the wireless network 4.

The input unit 23 comprises operating switches not shown and a touch panel, for example, disposed to the display 16, and inputs user 7 instructions by detecting user 7 operations.

The storage 24 nonvolatilely stores control programs and data required by the gateway device 3 to execute processes.

The near-field communication unit 25 is a device that communicates wirelessly by near-field communication with the wearable device 2, for example, as controlled by the controller 21, and in this embodiment communicates by Bluetooth®, wireless LAN, or ZigBee®.

The display 26 has an LCD panel, OLED panel, or other type of display panel, and displays information for the user 7 as controlled by the controller 21. The audio output unit 27 includes a microphone or other audio input device, and audio output device including an amplifier and speaker, and picks up and detects speech from the user 7, and outputs audio to the user 7, as controlled by the controller 21.

The position acquisition unit 28 is a device that acquires the current location using GPS, receives radio signals from GPS satellites through a GPS antenna not shown, and calculates the current position from latitude and longitude. Note that this embodiment uses the GPS system as an example, but positioning detection methods using other than the GPS system may be used.

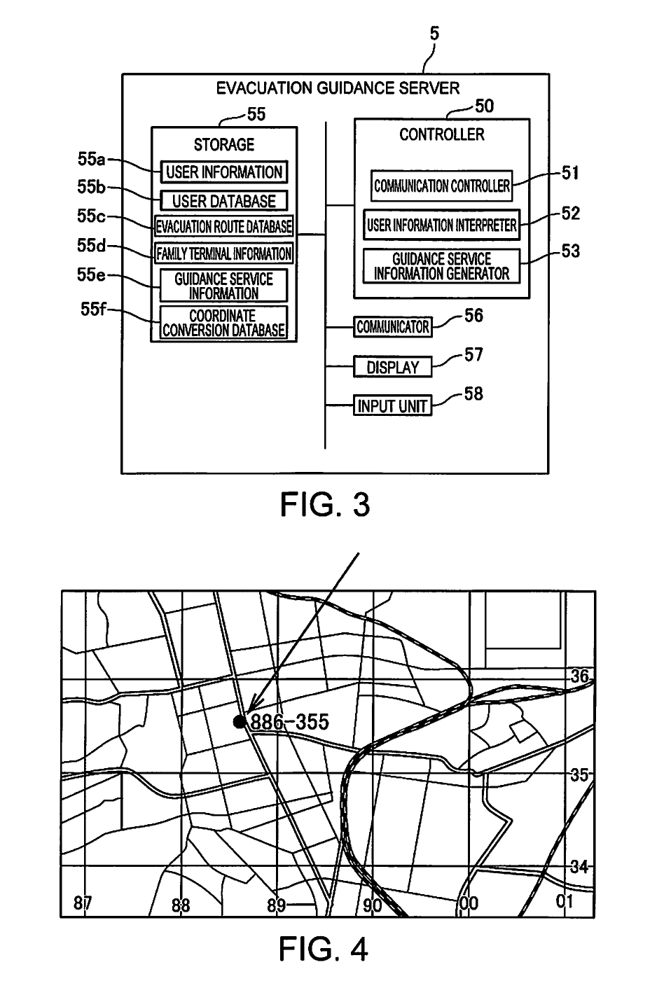

FIG. 3 is a function block diagram of the evacuation guidance server 5.

The evacuation guidance server 5 has a controller 50, storage 55, communicator 56, display 57, and input unit 58. Note that the evacuation guidance server 5 is not limited to configurations having only a single server device. For example, the functions of the evacuation guidance server 5 described below may be distributed to multiple server devices, and the evacuation guidance server 5 may be configured as a so-called cloud server of an unknown device configuration.

The controller 50 has the configuration of a computer including a CPU, ROM, and RAM. The controller 50 functions as an information processor that centrally controls other parts and operating processes by executing a basic control program stored in ROM, and other programs stored in storage 55.

The communicator 56 communicates as controlled by the controller 50 according to a specific wireless communication network standard, for example. The communicator 56 communicates with the disaster reporting server 6 through the wireless network 4, and receives disaster reports broadcast by the disaster reporting server 6 (FIG. 1). The communicator 56, by communicating with the gateway device 3 through the wireless network 4, also communicates with wearable devices 2 using the relay function of gateway devices 3. The communicator 56 is an example of a user information receiver and a guidance service information transmitter.

The display 57 has an LCD panel, OLED panel, or other type of display panel, and displays information for the user 7 as controlled by the display 57.

The input unit 58 includes a keyboard, mouse, or other input devices not shown, and detects operations by an operator not shown.

The storage 55 nonvolatilely stores control programs and data required by the evacuation guidance server 5 to execute processes. The storage 55 stores user information 55a, a user database 55b, an evacuation route database 55c, family terminal information 55d, guidance service information 55e, and a coordinate conversion database 55f.

The user information 55a is the user information the communicator 56 receives from the wearable device 2 and stores as controlled by the communication controller 51.

The user database 55b is a database storing information related to users 7 that use the guidance system 1. The user database 55b contains information enabling identifying users 7, such as an ID individually assigned to each wearable device 2. The user database 55b may also include the name, address, date of birth, and other information about the user 7. The user database 55b may also store, as information related to the user 7, the same information related to the body of the user that is contained in the physiological information 14c described above (FIG. 2A).

The evacuation route database 55c is a database storing evacuation facilities to which users 7 may be guided for refuge, and information required to determine evacuation routes to the evacuation facilities. The evacuation route database 55c also stores, for each evacuation facility, information such as the location, name, and who to contact (such as telephone numbers, e-mail addresses, SNS (social network service) accounts). The evacuation route database 55c also includes, for each evacuation facility, information indicating the types, or the types and volume, of medical services that can be provided.

The evacuation route database 55c includes map data for creating evacuation routes. The map data includes data related to the connections between routes that the user 7 may travel, and the locations of intersections. The map data may also include data indicating the class of vehicle that can travel a particular route, or if a particular route is a sidewalk or pedestrian path, and data indicating if the route is a road designated by law as closed to traffic in emergencies. Data indicating, for each route, the difficulty of travel for a user 7 travelling on foot or by bicycle, which may be expressed by data quantifying (scoring) the level of difficulty, may also be included. This difficulty data may also include such data as the degree and length of the slope of the road, the rise and number of steps, and whether the route includes pedestrian-only or bicycle-only sections. Data indicating obstacles and the ease of passage when the user 7 moves with a wheelchair, cane, or other assistive device may also be stored. The evacuation route database 55c is also not limited to route information, and may include information related to bridges, railroad crossings, train stations, and other landmarks.

The controller 50 functions as a communication controller 51, user information interpreter 52, and guidance service information generator 53 by the CPU executing programs described above.

The communication controller 51 controls the communicator 56 to pass communications between the wearable device 2, gateway device 3, and disaster reporting server 6. When the communicator 56 receives user information from the wearable device 2, the communication controller 51 stores the received user information as user information 55a in the storage 55.

The user information interpreter 52 interprets the user information 55a received by the communicator 56 and stored in the storage 55. The user information interpreter 52 extracts conditions related to the destination evacuation facility and the evacuation route appropriate to the user 7 using the wearable device 2 that sent the user information 55a. These conditions may include the types of medical services required at the destination evacuation facility, the level of difficulty of the evacuation route the user 7 can tolerate, and the mode of travel when the user 7 evacuates.

The guidance service information generator 53 determines the evacuation facility and evacuation route with the highest degree of compatibility with the conditions extracted by the user information interpreter 52, generates guidance service information for guiding the user 7 to the selected evacuation facility and evacuation route, and stores the guidance service information 55e in the storage 55.

The user information interpreter 52 also interprets the user information received by the communicator 56, and extracts positioning information indicating the location of the user 7, and physiological information related to the body of the user 7. The user information interpreter 52 may store the extracted positioning information and physiological information in the user database 55b relationally to the ID of the user 7 or the ID of the wearable device 2.

The family terminal information 55d (terminal-device-related information) includes, for each wearable device 2 used in the guidance system 1, information identifying another wearable device 2 that is linked as a destination terminal to which related information is sent. When sending guidance service information to a wearable device 2, the communication controller 51 also sends the guidance service information or related information to the other wearable device 2 linked by the family terminal information 55d. As a result, multiple users 7 using multiple wearable devices 2 related by the family terminal information 55d can share information related to the evacuation of each individual. For example, if wearable device 2A and wearable device 2B are related by the family terminal information 55d, user 7A and user 7B can share the content of their guidance service information.

The guidance service information 55e is evacuation-related information generated by the guidance service information generator 53 as described above, and is sent by the communication controller 51 to the wearable device 2. The guidance service information 55e may also include guidance service information of which the format and content may differ in part for transmission to another wearable device 2 related by the family terminal information 55d to the target wearable device 2. For example, guidance service information 55e sent to the wearable device 2 that is the intended destination of the guidance service information is evacuation-related information informing the user 7 of the evacuation facility and appropriate evacuation route. However, the information sent to another wearable device 2 that is linked by the family terminal information 55d may be limited to only information related to the evacuation facility sent to the first wearable device 2.

The coordinate conversion database 55f is a database storing information for converting positioning information expressed by latitude and longitude to UTM grid coordinates as described below.

When a disaster occurs, the guidance system 1 preferably reports the accurate location of the user 7, and provides an evacuation route based on the accurate location. As a result, UTM grid coordinates are used to accurately determine the location of the user 7 in a disaster.

UTM grid coordinates are described next.

The UTM grid is known internationally as the MGRS (Military Grid Reference System), and is a method of determining a position on Earth, and the format of positioning information expressed by this method. As described below, an advantage of using the UTM grid is that a position can be expressed more easily than with latitude and longitude coordinates.

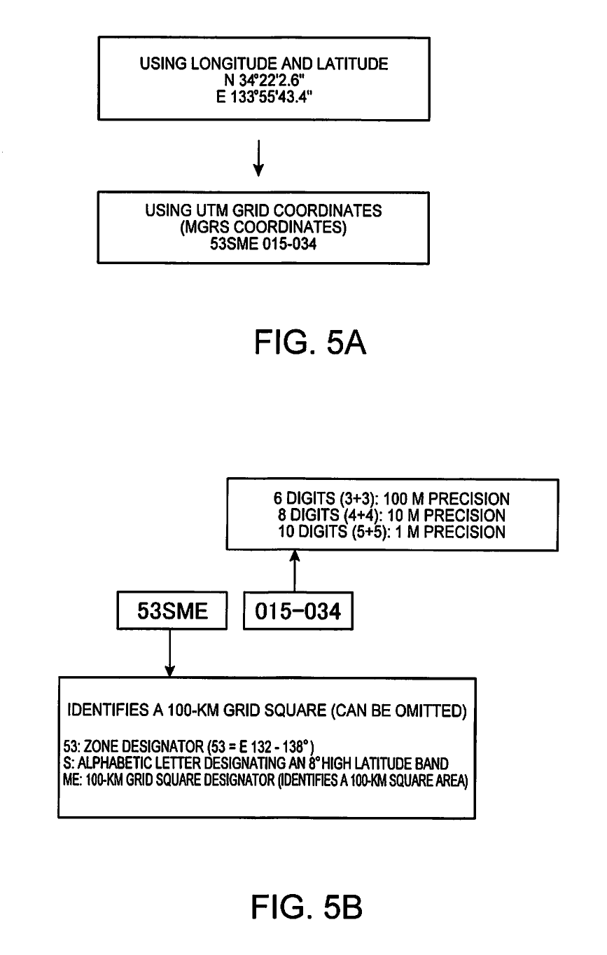

FIG. 4 shows an example of a UTM grid map applying the UTM grid to a map. FIG. 5A illustrates the relationship between UTM grid coordinates and latitude and longitude, and FIG. 5B illustrates the format of the UTM grid coordinates.

A UTM Grid map is a map that divides a map created as a Universal Transverse Mercator (UTM) conformal projection into units of a specific length on the north-south axis and east-west axis. A UTM Grid map has a grid (standard lines) extending north-south and east-west at specific intervals, and uses coordinates to identify specific areas (grid zones) in the grid. These coordinates are called UTM Grid coordinates.

In a typical UTM Grid, a target area (which may be the entire Earth) is divided into grid squares a maximum 100 km per side, and the 100 km grid squares are further divided into a grid of smaller areas at a 100 m, 10 m, or 1 m precision. UTM grid coordinates have a hierarchical structure combining a 5-digit area code identifying the largest grid square, and a code of a specific number of digits identifying a position within the grid square.

FIG. 5B shows 53SME as an example of a 5-digit area code identifying a 100 km grid square. In this area code, 53 is the zone number indicating the east-west position. The latitude (north-south direction) is divided into bands 8° high, an alphabetic letter is assigned to each band, and in this example the S in the area code identifies the latitude band. The ME in the area code is a code identifying this area code is a 100-km UTM grid square code specifying 100-km square area. The east-west zone number identifies a longitudinal zone (in the example in the figure, 132-138° east longitude), and the code indicating the north-south position corresponds to a latitude band of a specific height (8° latitude in the example in the figure). The area code can be omitted. For example, to express a position anywhere in Japan using UTM grid coordinates, the area code is required because the target area is significantly larger than the 100 km square size of the maximum grid square. However, to identify a location in an area contained within a 100 km square, every position will have the same 5-digit area code, and the area code can be omitted.

The square areas into which grid squares are divided at 100 m, 10 m, or 1 m units are identified by an n-digit number (where n is an integer) identifying the north-south position, and an n-digit number identifying the east-west position. This number of two n-digit values may also be referred to as MGRS coordinates. For example, the UTM grid coordinates of the area including the position indicated by the arrow in FIG. 4 are 886-355. In the example in FIG. 5A, the position identified by the longitude and latitude coordinates 34° 22′2.6″N and 133° 55′43.4″E is identified by the UTM grid coordinates 53SME 015-034.

If the grid interval is 100 m, the UTM grid coordinates are a 6-digit value where n=3. If the grid interval is 10 m, the UTM grid coordinates are an 8-digit value where n=4. If the grid interval is 1 m, the UTM grid coordinates are a 10-digit value where n=5.

The UTM grid coordinates thus express an area including a specific position (location), and the precision is determined by the size of the grid (100 m, 10 m, or 1 m). If the UTM grid coordinates are a 6-digit value, such as 015-034, an area 100 m square can be identified, or in other words, the precision is 100 m. The precision is 10 m if the coordinates are 8 digits long (4+4 digits), and the precision is 1 m if the coordinates are 10 digits long (5+5 digits). Increasing the number of digits in the coordinates enables identifying a location with high precision, and if high precision is not required, the number of digits in the coordinates can be reduced.

To express the current location in latitude and longitude using a 5 digit value for example, three digits are required in the integer portion, and the decimal portion is two digits. Because 1° of latitude and longitude is approximately equal to 111 km (40000 km/360°), a 2-digit decimal portion means a precision of approximately 1.11 km. More digits are required to express a location with greater precision, and if the decimal portion is 4 digits (the total length is 7 digits), the precision is approximately 11 m.

In other words, if a method of identifying a location by latitude and longitude is used, and a certain degree of precision is required, a long string of many digits including seven or more digits expressing the latitude and seven or more digits expressing the longitude is required. Communicating such a long string of numbers verbally invites communication errors.

For the foregoing reason, using grid coordinates is extremely effective. More specifically, a UTM coordinate value using a combination of 4+4 digits can express a position with the same precision as latitude and longitude coordinates of 7+7 digits.

An advantage of using UTM grid coordinates is that less information is required to express the same position than with latitude and longitude coordinates, and the expression is simpler. In the examples shown in FIG. 5A and FIG. 5B, the area code 53SME can be omitted, the coordinates expressed as only 015-034, and the position identified by this 6-digit value can be identified with 100 m precision. Because the coordinate structure is simple, the coordinates can be easily and accurately communicated from person to person, which is particularly useful when communicating a location by telephone or radio. If a UTM grid map and UTM grid coordinates are used, even someone that is unfamiliar with local place names or addressing methods can easily identify and know a position. In places with place names that are difficult to read or have complicated administrative divisions, place names and addresses can be difficult for someone from a different place to understand. When a disaster occurs, it is therefore desirable for people involved in rescue operations in the disaster zone to be able to use a method that does not depend on place names and addresses to identify a specific location in the disaster zone. A method that does not use place names and addresses is particularly desirable in the case of a wide area disaster or search and rescue operation requiring rescue workers from outside the local area. UTM grid coordinates are therefore an extremely effective tool for communicating positioning information because locations can be identified more reliably using few digits.

The location information contained in the user information 55a identifies the position the wearable device 2 detected using the GPS system, for example, and more specifically is positioning information expressed by latitude and longitude. The user information interpreter 52 therefore converts the positioning information contained in the user information 55a to UTM grid coordinates based on the coordinate conversion database 55f.

The evacuation route database 55c used by the guidance service information generator 53 to determine the evacuation route includes UTM grid coordinate information. More specifically, the locations of evacuation facilities that can be used for refuge, the locations of roads, and the locations of bridges, railroad crossings, train stations, and other landmarks include the UTM grid coordinates identifying the locations.

The guidance service information generator 53 can therefore determine the location of the wearable device 2 by UTM grid coordinates, and based on those UTM grid coordinates, can select an evacuation destination suitable for the user 7, and determine the evacuation route to that evacuation site.

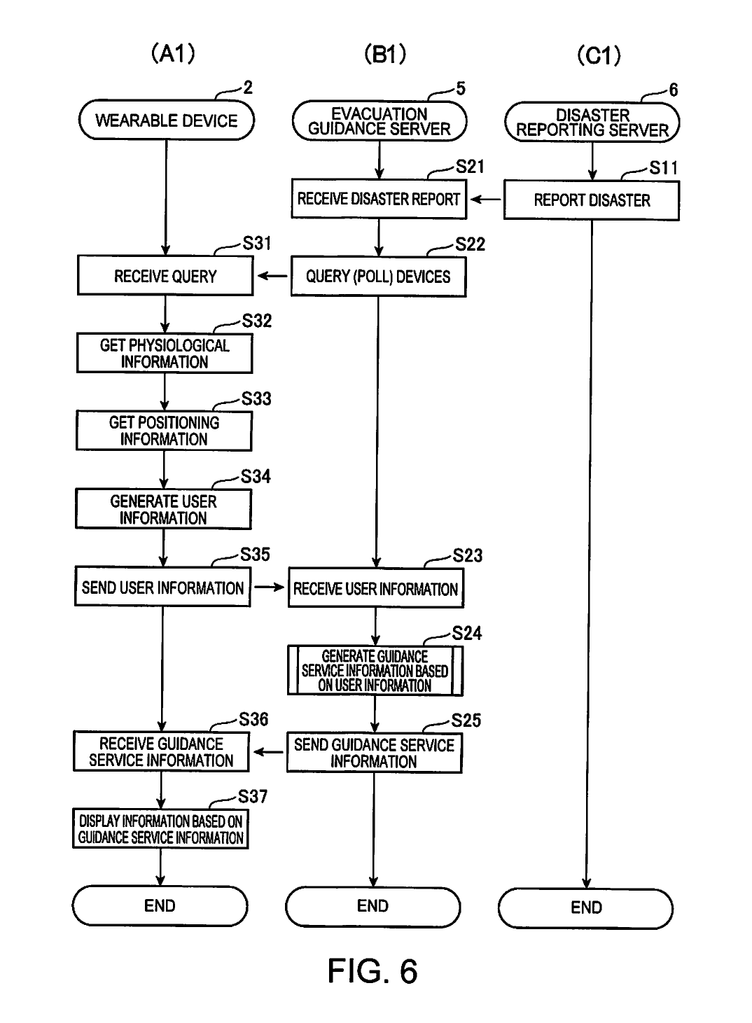

FIG. 6 is a flow chart of the operation of the guidance system 1. Column A1 shows the operation of the wearable device 2, column B1 shows the operation of the evacuation guidance server 5, and column C1 shows the operation of the disaster reporting server 6.

In the operation shown in FIG. 6, the evacuation guidance server 5 operates in response to receiving a disaster report from the disaster reporting server 6. The disaster reporting server 6 sends a disaster report to the evacuation guidance server 5 based on disaster information reported automatically by a meteorological monitoring system or disaster information input by a person (step S11).

The evacuation guidance server 5 receives the report from the disaster reporting server 6 and detects a disaster (step S21), and then queries (polls) the wearable device 2 (step S22). The devices polled in step S22 by the evacuation guidance server 5 may be all wearable devices 2 connected to the communication network of a mobile communication carrier, for example, instead of being limited to devices in a specific region. In this case, wearable devices 2 capable of communicating through the wireless network 4 are polled by the evacuation guidance server 5. The evacuation guidance server 5 may also poll wearable devices 2 in a specific area by specifying relay devices 4a, 4b connected to the wireless network 4, or other relay devices. This enables focusing on the area in which there are expected to be disaster victims, and on wearable devices 2 located in the area where evacuation is required, and greater efficiency can therefore be expected.

When polled by the evacuation guidance server 5 (step S31), the wearable device 2 starts the process of sending user information. Note that the wearable device 2 may determine if the sensor unit 12 is touching the body of the user 7, and execute the operation described below if the sensor unit 12 is touching the body of the user 7. For example, the wearable device 2 may not respond to polling by the evacuation guidance server 5 if contact of the sensor unit 12 with the skin of the user 7 cannot be detected.

The wearable device 2 responds to polling, and acquires the physiological information (step S32). In step S32, bio-information or physiological information 14c is acquired by the sensor unit 12. The wearable device 2 also acquires positioning information (step S33). In step S33, the location of the wearable device 2 is detected by the position acquisition unit 18 using the GPS system, or positioning information for the position detected by the gateway device 3, for example, is acquired.

Based on the information acquired in step S32 and S33, the wearable device 2 generates user information 14d (step S34), and sends the user information 14d to the evacuation guidance server 5 (step S35).

The evacuation guidance server 5 receives the user information sent by the wearable device 2 (step S23), and by interpreting the received user information, executes a guidance service information generating process to generate evacuation information appropriate to the user 7 (step S24). Next, the evacuation guidance server 5 sends the generated guidance service information to the wearable device 2 (step S25).

The wearable device 2 receives and stores the guidance service information sent by the evacuation guidance server 5 in the storage 14 as guidance service information 14e (step S36). Based on the guidance service information 14e, the wearable device 2 displays information contained in the guidance service information 14e (step S37).

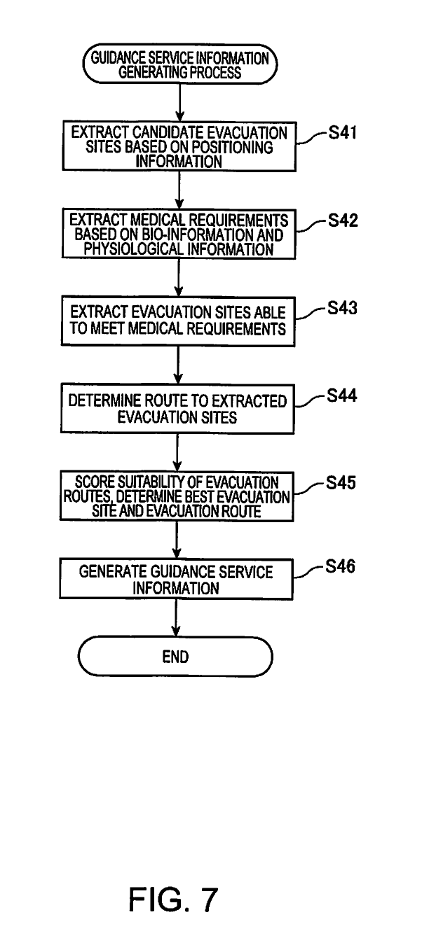

FIG. 7 is a flow chart of steps in the guidance service information generating process executed in step S24 in column B1.

Based on the positioning information contained in the user information 55a, the evacuation guidance server 5 references the evacuation route database 55c and extracts evacuation facility candidates from the evacuation facilities that can be used for refuge (step S41). In this example, the evacuation guidance server 5 converts the positioning information contained in the user information 55a to UTM grid coordinates, and then executes step S41.

The evacuation guidance server 5, based on the bio-information and/or physiological information contained in the user information 55a, extracts medical conditions related to the evacuation (step S42).

From the candidate evacuation facilities extracted in step S41, the evacuation guidance server 5 extracts the evacuation facilities that meet the conditions extracted in step S42 (step S43). Multiple evacuation facilities may be extracted in step S43. The evacuation guidance server 5 then determines an evacuation route to each of the evacuation facilities extracted in step S43 (step S44). If multiple evacuation facilities are extracted in step S43, or there are multiple evacuation routes to one evacuation facility from the location of the wearable device 2, multiple evacuation routes are determined in step S44.

The evacuation guidance server 5 then scores the suitability of each evacuation route determined in step S44 for evacuation of the user 7, and determines the destination and evacuation route for guiding the user 7 (step S45). The suitability of the evacuation routes is determined, for example, so that the difficulty of actual evacuation by the user 7 is not excessive. More specifically, the distance to the evacuation facility from the location identified by the positioning information in the user information, the means of travel used by the user 7, degree and length of the slope of the road, the rise and number of steps, and whether the route includes pedestrian-only or bicycle-only sections, are scored. Whether the route includes roads or bridges that may be made impassable by the disaster is also scored. Information related to roads or bridges that may be made impassable by the disaster are previously identified by a disaster hazard map compiled by a government organization and included in the evacuation route database 55c. Parameters, tables, formulae, and other information for calculating a score from the evacuation route information may also be included in the evacuation route database 55c. In step S45, a score indicating the degree of difficulty for the user 7 is calculated for each evacuation route determined in step S43. The evacuation guidance server 5 determines the evacuation route based on the calculated scores. For example, the evacuation route scored to have the lowest degree of difficulty could be selected as the evacuation route. Note that if the evacuation route scoring process includes calculating and evaluating the relationship between the positioning information sent by the wearable device 2 and the evacuation facilities, such as the distance, the positioning information may be expressed by latitude and longitude instead of UTM grid coordinates.

Next, the evacuation guidance server 5 generates guidance service information providing guidance to the evacuation route and evacuation facility determined in step S45 (step S46), and then returns to column B1.

As described above, the guidance location can be selected and changed based on the physiological information of the user 7, and guidance service information appropriate to the user 7 can be generated and provided by the guidance service information generating process. For example, the location to which the user 7 is guided could be changed based on the blood type of the user 7. Based on the vital information, guidance service information instructing evacuating and moving to a facility (such as a hospital) where medical staff are always available can be generated and sent to a user 7 identified as a person that requires immediate medical attention. In addition, based on the vital information, guidance service information instructing evacuating and moving to a common evacuation site can be generated and sent to users 7 identified as not requiring medical attention. The evacuation guidance server 5 and guidance system 1 can adapt and provide guidance appropriate to the specific situation of the user 7.

In the guidance service information generating process in FIG. 7, the evacuation guidance server 5, based on the user information 55a, detects the health and medical status of the user 7 to determine the destination and evacuation route appropriate to evacuation of the user 7. In the guidance service information generating process, the evacuation guidance server 5 calculates the acceptance capacity of each evacuation facility, and may determine the appropriateness of the guidance service information based on the calculated acceptance capacity. The evacuation guidance server 5 may also be configured to generate evacuation information for guiding to another evacuation site and evacuation route based on the score if a facility is determined not appropriate. For example, the evacuation guidance server 5 may execute as necessary a process reflecting damage to each evacuation facility in the evacuation route database 55c. The evacuation guidance server 5 may also determine the evacuation destinations to match the number of people that can be accommodated by each evacuation facility with the number of users 7 guided to that evacuation facility as the destination. In this case, medical resources can be desirably distributed to evacuation facilities in the disaster zone.

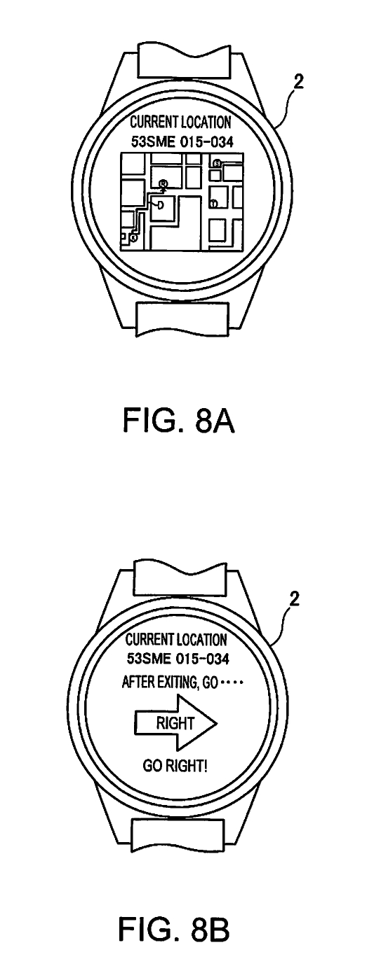

FIG. 8A and FIG. 8B show examples of displaying evacuation information on a wearable device 2, FIG. 8A showing an example displaying a map, and FIG. 8B as showing an example displaying the direction to proceed.

As shown in FIG. 8A, based on the guidance service information 14e received from the evacuation guidance server 5 and stored, the wearable device 2 displays on the screen of the display 16 a map including the location of the evacuation site and the evacuation route from the current location of the wearable device 2 to the evacuation site. To display the information as shown in FIG. 8A, the wearable device 2 may acquire the current location by the position acquisition unit 18, and display the current location overlaid on the map contained in the guidance service information 14e.

As shown in FIG. 8B, the wearable device 2 may display a directional arrow indicating the direction the user 7 is to proceed along the evacuation route. An advantage of the example in FIG. 8B is that the user 7 can be appropriately guided along the route to the evacuation site even when the display area of the wearable device 2 is relatively small.

To display information as shown in FIG. 8B, the wearable device 2 acquires the current location from the position acquisition unit 18, and displays guidance based on the acquired current location and the guidance service information 14e. The wearable device 2 may also detect the direction of the wearable device 2 by the geomagnetic sensor of the motion sensor 12b to display appropriate guidance.

As shown in FIG. 8A and FIG. 8B, the wearable device 2 can display the UTM grid coordinates of the current position. For example, when the evacuation guidance server 5 converts the position of the wearable device 2 to UTM grid coordinates based on the coordinate conversion database 55f, and sends guidance service information including the converted UTM grid coordinates, the wearable device 2 can display the UTM grid coordinates based on the guidance service information 14e. In this case, there is no need for a process on the wearable device 2 to convert coordinates to the UTM grid. By displaying UTM grid coordinates on the display of the wearable device 2, the user 7 can, for example, know the UTM grid coordinates of his current location from the display of the wearable device 2. Users 7 can therefore quickly and easily report their current location when requesting help by telephone.

Note that to display the evacuation route as shown in FIG. 8A or FIG. 8B, the controller 11 of the wearable device 2 may also control the audio output unit 17 to report the UTM grid coordinates and evacuation route by artificial speech.

As described above, a guidance system 1 according to this embodiment includes a wearable device 2 used by a user 7, and an evacuation guidance server 5 capable of communicating with the wearable device 2. The wearable device 2 includes a physiological information acquisition unit, such as a sensor unit 12 that acquires physiological information about the user 7, and a position acquisition unit 18 that acquires positioning information indicating the location of the wearable device 2. The wearable device 2 sends user information including the acquired physiological information and the positioning information acquired by the position acquisition unit 18 by the near-field communication unit 15 through the gateway device 3 to the evacuation guidance server 5. The evacuation guidance server 5 has a communicator 56 that receives the user information sent by the wearable device 2. The evacuation guidance server 5, based on the user information received by the communicator 56, generates guidance service information related to evacuation appropriate to the wearable device 2 by means of the guidance service information generator 53 of the controller 50. The controller 50 sends the generated guidance service information by the communicator 56 to the wearable device

The communicator 56 of the evacuation guidance server 5 receives positioning information indicating the position of the wearable device 2, and user information including the physiological information of the user 7, from the wearable device 2. Based on the user information the communicator 56 receives, the evacuation guidance server 5 generates guidance service information related to evacuation appropriate to the wearable device 2, and sends the guidance service generated by the controller 50 to the wearable device

In the information processing method of the foregoing embodiment, the wearable device 2 acquires the physiological information of the user 7, acquires positioning information indicating the location of the wearable device 2, and sends user information including the physiological information and positioning information to the evacuation guidance server 5. The evacuation guidance server 5 receives the user information the wearable device 2 sent, and based on the user information, generates guidance service information related to evacuation for the wearable device 2, and sends the guidance service information to the wearable device 2.

The guidance system 1 and information processing method of the guidance system 1, when evacuation or rescue is required, enable sending to the wearable device 2 of the user 7 guidance service information that is generated with consideration for the location of the wearable device 2 and the physiological information of the user 7. As a result, appropriate guidance service information can be provided to the user 7, and support can be provided quickly and efficiently to multiple users 7.

In the guidance system 1, the wearable device 2 has a physiological information acquisition unit, such as a sensor unit 12, that acquires physiological information about the user 7 wearing the wearable device 2, and a position acquisition unit 18 that acquires positioning information indicating the position of the wearable device 2. The wearable device 2 sends user information including the acquired physiological information and positioning information acquired by the position acquisition unit 18 to the evacuation guidance server 5. The wearable device 2 also receives guidance service information sent from the evacuation guidance server 5 based on the transmitted user information. The wearable device 2 may be configured with a contact sensor 12a that contacts the skin of the user 7, and using the wearable device 2 may be enabled when the contact sensor 12a is touching the skin of the user 7. In this case, there is no need to send guidance service information to a wearable device 2 that the user 7 is not wearing, and the overall operating efficiency of the guidance system 1 can be improved.

The wearable device 2 detects bio-information through the contact sensor 12a, and acquires the detected bio-information as physiological information; and the controller 50 generates guidance service information based on the physiological information contained in the user information received by the communicator 56. As a result, bio-information of the user 7 is sent from the wearable device 2 to the evacuation guidance server 5, and guidance service information is generated based on the bio-information. As a result, guidance service information reflecting the physical condition of the user 7 can be provided to the user 7. Information related to the physical condition of the user 7 of the wearable device 2 can also be accumulated on the evacuation guidance server 5, and information useful for determining the scale and condition of human suffering when a disaster occurs can be acquired.

The evacuation guidance server 5 also stores family terminal information 55d relating the wearable device 2 to other wearable devices 2. The controller 50 can also include, in the guidance service information that is sent to the target wearable device 2, information related to other wearable devices 2 that are linked by the family terminal information 55d to that target wearable device 2. More specifically, information related to other wearable devices 2 that are linked by the family terminal information 55d can be sent to the destination wearable device 2 to which guidance service information is sent in step S25 (column B1). In this case, information related to the users 7 of other related wearable devices 2 can be sent to the user 7 of the target wearable device 2.

The information sent to the related wearable devices 2 may be the same as the guidance service information transmitted in step S25. Information related to the user 7 and the wearable device 2 to which guidance service information was sent in step S25 may be information that is added to guidance service information. That is, the controller 50, based on user information the communicator 56 receives from the wearable device 2, may generate guidance service information for the other wearable devices 2. In this case, guidance service information reflecting the situation of the user 7 of another related wearable device 2 can be provided to the user 7 of the wearable device 2.

Furthermore, when sending guidance service information to a wearable device 2, the evacuation guidance server 5 may also send the guidance service information to a previously specified other destination. More specifically, guidance service information may be sent to the evacuation facility terminal 8 (FIG. 1) of the evacuation facility selected as the destination to which the user 7 is instructed to evacuate. In this case, the evacuation facility can get information about users 7 expected to evacuate to that facility. If physiological information in the user information 55a is included in the guidance service information sent to the evacuation facility terminal 8, information related to medical services the evacuating user 7 may require can be provided to the evacuation facility. The evacuation facility can therefore prepare more efficiently to receive users 7. Furthermore, if the user 7 does not arrive by the time the user 7 is expected to arrive, appropriate action, such as starting a search operation, can be taken.

The wearable device 2 can also detect bio-information through the contact sensor 12a, and acquire the detected bio-information as physiological information. As a result, bio-information detected by contact with the body of the user 7 can be sent from the wearable device 2 to the evacuation guidance server 5, and guidance service information based on the bio-information can be generated. As a result, guidance service information reflecting the physical condition of the user 7 can be provided to the user 7. Furthermore, when the guidance system 1 is configured to pair wearable devices 2 with an evacuation guidance server 5, information related to the physical condition of the users 7 of the wearable devices 2 can be collected, and information useful for determining the scale and condition of human suffering when a disaster occurs, for example, can be acquired.

The wearable device 2 also has storage 14 that stores physiological information 14c related to the body of the user 7, and can acquire physiological information based on the physiological information 14c. In this case, guidance service information reflecting information related to the body of the user 7 can be provided to the user 7. For example, if the physical characteristics include information medical information, guidance service information appropriate to the medical needs of an individual user 7 can be provided to that user 7. Furthermore, because the wearable device 2 can send to the evacuation guidance server 5 positioning information detected by the position acquisition unit 18 and indicating the most recent position of the wearable device 2, more appropriate guidance service information can be provided to the user 7.

The controller 50 generates and sends to the wearable device 2 guidance service information including positioning information in a format defined for the evacuation guidance server 5, such as UTM grid coordinates. As a result, positioning information in a format for the evacuation guidance server 5 can be included in the guidance service information generated based on the positioning information the wearable device 2 sent, and sent to the wearable device 2. As a result, both the evacuation guidance server 5 and wearable device 2 can use positioning information in a different format than the positioning information the wearable device 2 sends to provide information related to user 7 evacuation. Therefore, positioning information suitable for providing evacuation information can be used without being limited by the specifications of the positioning information the wearable device 2 detects.

Furthermore, based on the guidance service information, the wearable device 2 can display information related to the destination and evacuation route on the display screen of the display 16. In this instance, the wearable device 2 can display positioning information in the format defined by the evacuation guidance server 5, such as UTM grid coordinates. As a result, positioning information in a different format than the positioning information the wearable device 2 sends can be used to provide information related to user 7 evacuation. Therefore, positioning information suitable for providing evacuation information can be used without being limited by the specifications of the positioning information the wearable device 2 detects.

The wearable device 2 has a power generator 19 that produces power, and a power supply 20 that supplies to the near-field communication unit 15 at least power generated by the power generator. As a result, the wearable device 2 shutting down due to insufficient power can be avoided, and the possibility of being able to use the wearable device 2 in a disaster can be improved.

The wearable device 2 has a motion sensor 12b that detects motion, and can send user information including the detection result of the motion sensor 12b to the evacuation guidance server 5. As a result, conditions related to the user 7 possessing the wearable device 2 can be detected indirectly based on movement of the wearable device 2, and guidance service information reflecting the condition of the user 7 can be provided to the user 7.

### Embodiment 2

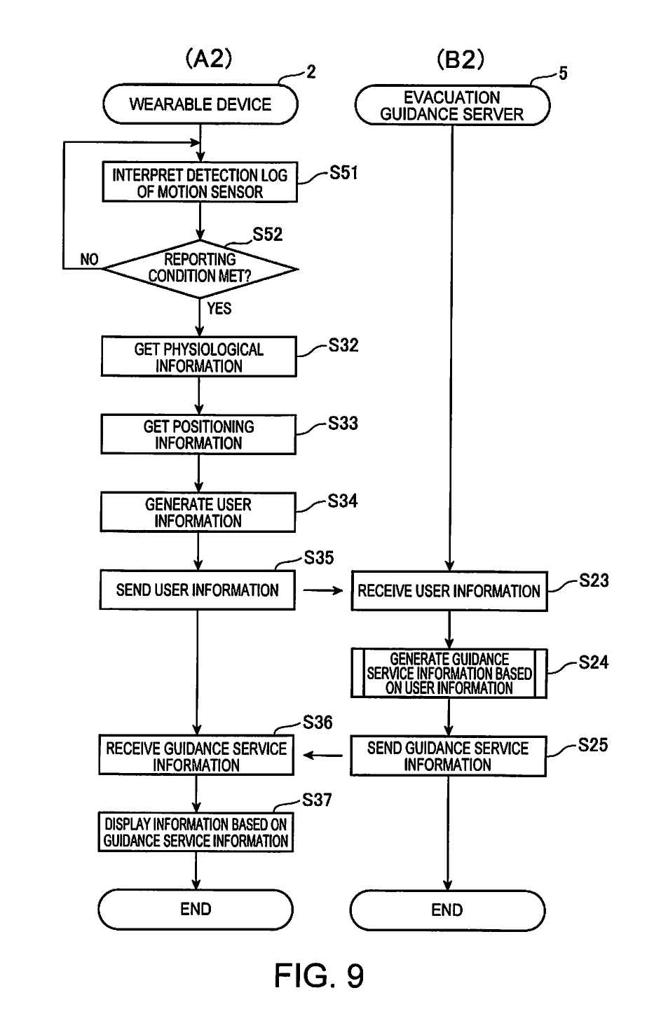

FIG. 9 is a flow chart of the operation of the guidance system 1 in a second embodiment of the invention, column A2 showing the operation of the wearable device 2, and column B2 showing the operation of the evacuation guidance server 5.

The configuration of a guidance system 1 according to the second embodiment of the invention is the same as the first embodiment, and further description of the configuration of the guidance system 1 is omitted in the figures and following description.

The first embodiment describes a configuration in which the wearable device 2 sends user information to the evacuation guidance server 5 as a result of the evacuation guidance server 5 polling the wearable device 2. This second embodiment of the invention describes a configuration in which the wearable device 2 sends user information to the evacuation guidance server 5 based on a value detected by the motion sensor 12b.

The wearable device 2 executes detection by the motion sensor 12b, and adds the detection value to a sensor detection log 14b as needed (FIG. 2A). The wearable device 2 then interprets the sensor detection log 14b (step S51), and determines if the detection value or change in the detection value of the sensor detection log 14b meets a previously set reporting condition (step S52).

A reporting condition is a condition for determining, based on the detection value or change in the detection value of the motion sensor 12b, if the user 7 is in a situation requiring evacuation, and is previously stored in the storage 14, for example. For example, if the user 7 has met with a natural disaster, such as an earthquake, torrential rain, landslide, or volcanic eruption, or an accident such as a traffic accident, the motion sensor 12b detects the acceleration or angular velocity from the movement of the wearable device 2 due to the effects of the disaster or accident. Whether or not the user 7 requires evacuation can therefore be determined based on whether or not the detection value or change in the detection value of the motion sensor 12b meets the reporting condition.

If the reporting condition is not met (step S52: No), the wearable device 2 returns to step S51, and continues interpreting the sensor detection log 14b at a previously set interval. If the reporting condition is met (step S52: Yes), the wearable device 2 goes to step S32. The operations of step S32 to S37 and step S23 to S25 are the same as described with reference to FIG. 6 and FIG. 7.

This second embodiment of the invention enables the user 7 to receive guidance service information provided by a evacuation guidance server 5 corresponding to the condition of the wearable device 2 even without being polled by the evacuation guidance server 5. As a result, the user 7 can receive evacuation support even in the event of a localized disaster or accident, for example.

Furthermore, if an input operation to the input unit 13 is an operation requesting evacuation, the wearable device 2 may execute the same operation when the detection value or change in the detection value of the motion sensor 12b meets the reporting condition (step S52: Yes). In this case, evacuation may be requested by a user 7 operation, and guidance service information provided by the evacuation guidance server 5 can be received.

### Embodiment 3

The configuration of a guidance system 1 according to the third embodiment of the invention is the same as the first embodiment, and further description of the configuration of the guidance system 1 is omitted in the figures and following description.

The first embodiment describes a configuration in which the wearable device 2 sends user information to the evacuation guidance server 5 as a result of the evacuation guidance server 5 polling the wearable device 2. This third embodiment of the invention describes a configuration adding generating and displaying a disaster map to the operation of the evacuation guidance server 5 described in the first embodiment (column B1).

In this embodiment, the near-field communication unit 15 is equivalent to a user information transmitter and terminal receiver. When the gateway device 3 relays wearable device 2 communications, the near-field communication unit 15 and gateway device 3 (particularly the communicator 22 and near-field communication unit 25 of the gateway device 3) work together as the user information transmitter and terminal receiver. In addition, the relay devices 4a, 4b connected to the wireless network 4 embody a communicator.

In addition, the contact sensor 12a embodies a bio-information detector, and by storing user information 55a, the storage 55 embodies a user information storage unit.

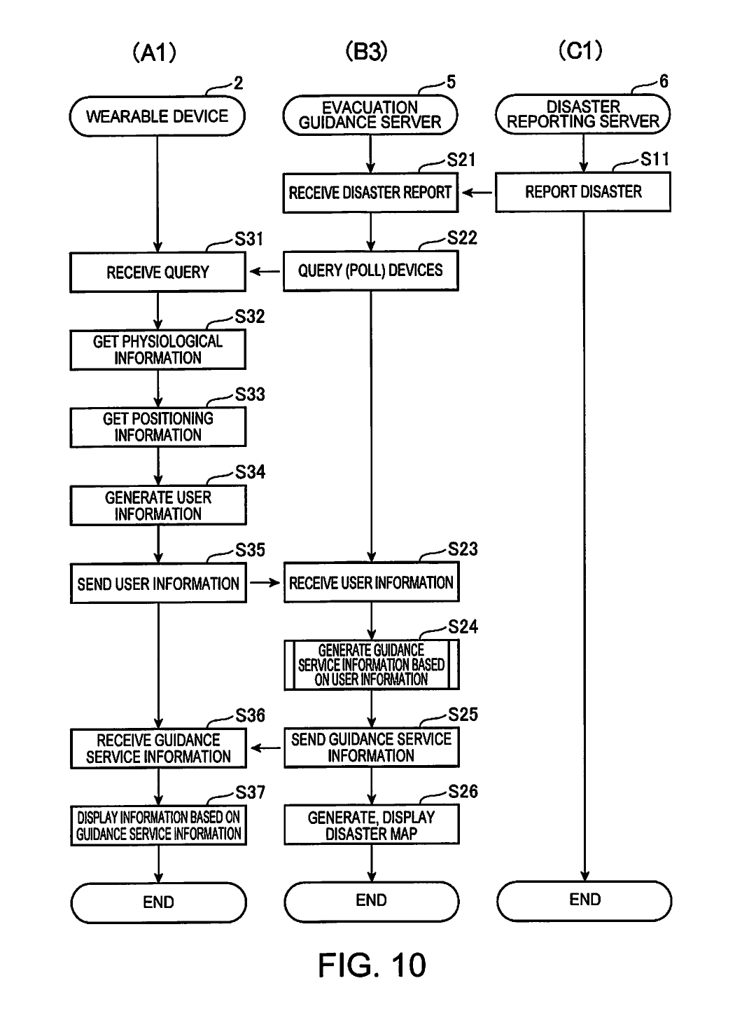

FIG. 10 is a flow chart of the operation of the guidance system 1. Column A1 shows the operation of the wearable device 2, column B3 shows the operation of the evacuation guidance server 5, and column C1 shows the operation of the disaster reporting server 6.

Note that in this embodiment column A1, column C1, and steps S21 to S25 in column B3 are the same as in the first embodiment, and further description thereof is omitted.

After sending the generated guidance service information to the wearable device 2 in step S25, the evacuation guidance server 5 in this embodiment generates and displays a disaster map on the display 57 (FIG. 3) based on the user information received in step S23 from the wearable device 2 (step S26).

In addition to user information received in step S23, the evacuation guidance server 5 may also use user information (positioning information, physiological information) received from the wearable device 2 or other wearable device 2 and stored in the storage 55.

The disaster map is information visually mapping the positioning information and physiological information contained in the user information, such as a map showing the location of the terminal device based on the positioning information. The map may be a planar coordinate map of UTM grid coordinates and markers or symbols showing the location of the wearable device 2. It may also be a map showing the marker or symbol for the location of the wearable device 2 overlaid to an actual map (such as a topographic map, town plan map, road map, administrative map, address map, or aerial photograph). Physiological information may also be indicated by using a color or shape representing particular physiological information as the marker or symbol indicating the location of the wearable device 2, or by a letter or symbol correlated to the marker or symbol indicating the location of the wearable device 2.

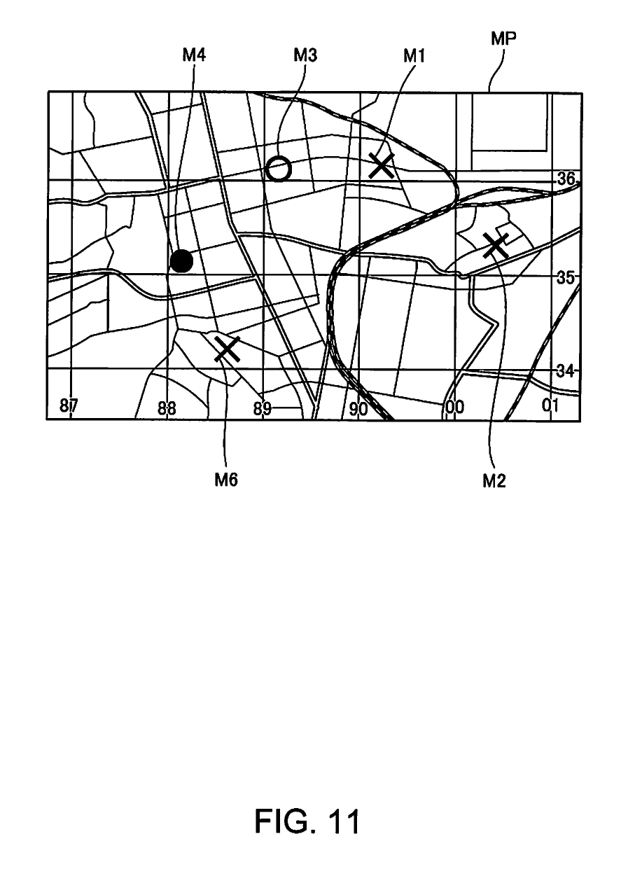

FIG. 11 shows an example of a disaster map generated and displayed by the evacuation guidance server 5.

The disaster map MP shown in FIG. 11 is a map overlaying (mapping) markers M1 to M6 based on user information to a road map. The latitude and longitude axes of the disaster map MP are displayed by the most significant two digits of the UTM grid coordinates for the longitude and latitude.

The markers M1 to M6 are placed according to the positioning information contained in the user information. In other words, the markers M1 to M6 indicate the position of the wearable device 2 that sent the user information. The markers M1 to M6 are of three types, ×, ●, and ◯, which correspond to physiological information contained in the user information. In this example, these types identify the user as a user requiring emergency medical service based on the physiological information, a user requiring non-urgent medical care, and a user that can evacuate on their own. The shapes of the markers M1 to M6 indicate the type of user using the corresponding wearable device 2.

By the display 57 of the evacuation guidance server 5 displaying the disaster map MP, the extend of the disaster, the number of users requiring evacuation or rescue, locations, and other information can be easily understood visually.

To compile the disaster map MP, the evacuation guidance server 5 polls the wearable devices 2 in step S22 in FIG. 10, and collects user information. In other words, the timing for collecting the user information used to create the disaster map MP is controlled by the evacuation guidance server 5. For example, if an indeterminate large number of wearable devices 2 dispersed over a wide area send user information at unexpected times, the evacuation guidance server 5 may take a long time to compile the disaster map MP, possibly overloading the processor. However, if the evacuation guidance server 5 can control polling to trigger input of user information, devices in a specific area can be polled and user information collected from wearable devices 2 in that area can be processed appropriately. The user information can then be used to compile a list or disaster map such as the disaster map MP.

As described above, a guidance system 1 according to this embodiment includes a wearable device 2 used by a user 7, and an evacuation guidance server 5 capable of communicating with the wearable device 2. The wearable device 2 includes a sensor unit 12 that acquires physiological information about the user 7, and a position acquisition unit 18 that acquires positioning information indicating the location of the wearable device 2. The wearable device 2 receives a query sent by the evacuation guidance server 5, and sends user information including the physiological information acquired by the sensor unit 12 and the positioning information acquired by the position acquisition unit 18 to the evacuation guidance server 5. The evacuation guidance server 5 sends a query to the wearable device 2, and receives user information from the wearable device 2.

The guidance system 1 and information processing method of the guidance system 1 thus comprised can cause the wearable device 2 to transmit information as controlled by the evacuation guidance server 5. As a result, because information related to the condition of the user 7 using the wearable device 2 can be collected by the information processing device, information can be collected quickly and efficiently from many users.

The evacuation guidance server 5 also has storage 55 that stores information combining the physiological information and positioning information contained in the user information received by the communicator 56. As a result, information collected from the wearable device 2 of a user 7 can be easily used. By interpreting or tabulating information received from the wearable devices 2 of users 7, the scale and state of damage can be determined in detail when a disaster occurs.

For example, the storage 55 may relationally store the physiological information and positioning information contained in the user information physiological information and positioning information may also be stored relationally to information unique (such as an ID) to the wearable device 2 that sent the user information, or information unique to the user information (such as a user information ID identifying the user information), physiological information and positioning information may also be stored relationally to information unique to the wearable device 2 that sent the user information, or the user of the wearable device 2. Further alternatively, physiological information and positioning information stored in the storage 55 may be in a format enabling processing in pairs.

The wearable device 2 also has a contact sensor 12a that touches the body of the user 7 and detects bio-information, and acquires the bio-information detected by the contact sensor 12a as physiological information, and transmits the physiological information by the near-field communication unit 15. As a result, extremely timely, more detailed information related to the physical status of the user 7 of the wearable device 2 can be collected.

The wearable device 2 also connects to an evacuation guidance server 5 through a wireless network 4 including relay devices 4a, 4b located strategically to the area. The evacuation guidance server 5 may specify the broadcast area for the relay devices 4a, 4b to poll wearable devices 2 in the specified area. In this case, wearable devices 2 in a specified area can be made to send information. As a result, information related to the status of users 7 using wearable devices 2 in a designated area can be collected by the evacuation guidance server 5.

The wearable device 2 detects bio-information through the contact sensor 12a, and acquires the detected bio-information as physiological information; and the controller 50, based on the physiological information contained in user information received by the communicator 56, generates guidance service information. As a result, bio-information of the user 7 is sent from the wearable device 2 to the evacuation guidance server 5, and based on the bio-information, guidance service information is generated. As a result, guidance service information reflecting the physical state of the user 7 can be provided to the user 7. Information related to the physical condition of the user 7 of the wearable device 2 can also be collected by the evacuation guidance server 5, and information useful for determining the scale and condition of human suffering when a disaster occurs can be acquired.

The evacuation guidance server 5 also stores family terminal information 55d relating the wearable device 2 to other wearable devices 2. The controller 50 can also include, in the guidance service information that is sent to the target wearable device 2, information related to other wearable devices 2 that are linked by the family terminal information 55d to that target wearable device 2. More specifically, information related to other wearable devices 2 that are linked by the family terminal information 55d can be sent to the destination wearable device 2 to which guidance service information is sent in step S25 (column B3). In this case, information related to users 7 of other related wearable devices 2 can be sent to the user 7 of the target wearable device 2.

The information sent to the related wearable devices 2 may be the same as the guidance service information transmitted in step S25. Information related to the user 7 and destination wearable device 2 to which guidance service information was sent in step S25 may be information that is added to guidance service information. That is, the controller 50, based on user information the communicator 56 receives from the wearable device 2, may generate guidance service information for the other wearable devices 2. In this case, guidance service information reflecting the situation of the user 7 of another related wearable device 2 can be provided to the user 7 of the wearable device 2.

Furthermore, when sending guidance service information to a wearable device 2, the evacuation guidance server 5 may also send the guidance service information to a previously specified other destination. More specifically, guidance service information may be sent to the evacuation facility terminal 8 (FIG. 1) of the evacuation facility selected as the destination to which the user 7 is instructed to evacuate. In this case, the evacuation facility can get information about users 7 expected to evacuate to that facility. If physiological information in the user information 55a is included in the guidance service information sent to the evacuation facility terminal 8, information related to medical services the evacuating user 7 may require can be provided to the evacuation facility. The evacuation facility can therefore prepare more efficiently to receive users 7. Furthermore, if the user 7 does not arrive by the time the user 7 is expected to arrive, appropriate action, such as starting a search operation, can be taken.

The wearable device 2 can also detect bio-information through the contact sensor 12a, and acquire the detected bio-information as physiological information. As a result, bio-information detected by contact with the body of the user 7 can be sent from the wearable device 2 to the evacuation guidance server 5, and guidance service information based on the bio-information can be generated. As a result, guidance service information reflecting the physical condition of the user 7 can be provided to the user 7. Furthermore, when the guidance system 1 is configured to pair wearable devices 2 with an evacuation guidance server 5, information related to the physical condition of the users 7 of the wearable devices 2 can be collected, and information useful for determining the scale and condition of human suffering when a disaster occurs, for example, can be acquired.

The wearable device 2 also has storage 14 that stores physiological information 14c related to the body of the user 7, and can acquire physiological information based on the physiological information 14c. In this case, guidance service information reflecting information related to the body of the user 7 can be provided to the user 7. For example, if the physical characteristics include information medical information, guidance service information appropriate to the medical needs of an individual user 7 can be provided to that user 7. Furthermore, because the wearable device 2 can send to the evacuation guidance server 5 positioning information detected by the position acquisition unit 18 and indicating the most recent position of the wearable device 2, more appropriate guidance service information can be provided to the user 7.

The controller 50 generates and sends to the wearable device 2 guidance service information including positioning information in a format defined for the evacuation guidance server 5, such as UTM grid coordinates. As a result, positioning information in a format for the evacuation guidance server 5 can be included in the guidance service information generated based on the positioning information the wearable device 2 sent, and sent to the wearable device 2. As a result, both the evacuation guidance server 5 and wearable device 2 can use positioning information in a different format than the positioning information the wearable device 2 sends to provide information related to user 7 evacuation. Therefore, positioning information suitable for providing evacuation information can be used without being limited by the specifications of the positioning information the wearable device 2 detects.

Furthermore, based on the guidance service information, the wearable device 2 can display information related to the destination and evacuation route on the display screen of the display 16. In this instance, the wearable device 2 can display positioning information in the format defined by the evacuation guidance server 5, such as UTM grid coordinates. As a result, positioning information in a different format than the positioning information the wearable device 2 sends can be used to provide information related to user 7 evacuation. Therefore, positioning information suitable for providing evacuation information can be used without being limited by the specifications of the positioning information the wearable device 2 detects.

The wearable device 2 has a power generator 19 that produces power, and a power supply 20 that supplies to the near-field communication unit 15 at least power generated by the power generator. As a result, the wearable device 2 shutting down due to insufficient power can be avoided, and the possibility of being able to use the wearable device 2 in a disaster can be improved.

The wearable device 2 has a motion sensor 12b that detects motion, and can send user information including the detection result of the motion sensor 12b to the evacuation guidance server 5. As a result, conditions related to the user 7 possessing the wearable device 2 can be detected indirectly based on movement of the wearable device 2, and guidance service information reflecting the condition of the user 7 can be provided to the user 7.

The foregoing examples describe preferable embodiments of the invention, but do not limit the scope of the invention, and can be varied in many ways without departing from the scope of the accompanying claims. For example, the specific format of the guidance service information the evacuation guidance server 5 provides to the wearable device 2 in the foregoing embodiments may be configured as desired, and may include map information. The wearable device 2 may also be configured to store map information in storage 14, and display a map on the display 16 based on the map information. In this case, there is no need to include map information in the guidance service information the evacuation guidance server 5 sends, and the amount of data that is exchanged can be reduced. Furthermore, information of the destination included in the guidance service information may be a code previously assigned to the evacuation site, and in this case the wearable device 2 may relationally store, in advance, information relating evacuation site codes to addresses and contact information for the evacuation sites. Further alternatively, the foregoing embodiments are configured so that the wearable device 2 or gateway device 3 detects the position by GPS as the positioning information. The invention is not so limited, however, and the position of the wearable device 2 or gateway device 3 may be detected by communicating wirelessly by Wi-Fi® and calculating the position relative to nearby Wi-Fi access points.

The wearable device 2 is also not limited to a wristwatch design that can be worn on the wrist of the user 7, and may be any configuration capable of communicating with the evacuation guidance server 5 or gateway device 3, displaying images, or making images visible to the user. More specifically, the wearable device 2 may be configured as eyeglasses, rings, or clothing, for example.

The function blocks of devices including the wearable device 2, gateway device 3, and evacuation guidance server 5 shown in FIG. 2A, FIG. 2B, and FIG. 3 represent functional configurations embodied by the cooperation of hardware and software, and the specific configuration of devices in the guidance system 1 is not limited to the foregoing block diagrams. Therefore, hardware corresponding individually to the function blocks shown in the function block diagrams is not required, and can obviously configured by a single processor embodying the functions of multiple function units by executing a program. Furthermore, some functions embodied by software in the foregoing embodiments may be embodied by hardware, and some functions embodied by hardware in the foregoing embodiments may be embodied by software. The specific detailed configuration of other parts of the guidance system 1 can also be modified as desired without departing from the scope of the accompanying claims.

An evacuation guidance server for providing evacuation guidance in a disaster is used as a specific example of an information processing device according to the invention in the foregoing embodiments, but the invention is not limited to providing evacuation guidance. The information processing system of the invention can accurately acquire health information of individual users by acquiring physiological information of the individual users using terminal devices. As a result, the invention can be used for a wide range of applications other than disasters, and is, for example, an effective technology for monitoring users, and is technology that is particularly effective in advanced societies with an aging population.

### REFERENCE SIGNS LIST

1 guidance system (information processing system)
2, 2A, 2B wearable device (terminal device)
3, 3A, 3B gateway device
4 communication network
5 evacuation guidance server (information processing device)
6 disaster reporting server
7, 7A, 7B user
8 evacuation facility terminal
11 controller
12 sensor unit (physiological information acquisition unit)
12a contact sensor (body contact unit)
12b motion sensor (motion detector)
13 input unit
14 storage (terminal storage)
14a location detection log
14b sensor detection log
14c physiological information (physical characteristics)
14d user information
14e guidance service information
15 near-field communication unit (user information transmitter, guidance service information receiver)
16 display (terminal display unit)
17 audio output unit
18 position acquisition unit (positioning information acquisition unit)
19 power generator
20 power supply (power supply unit)
21 controller
22 communicator
25 near-field communication unit
50 controller
51 communication controller
52 user information interpreter
53 guidance service information generator
55 storage
55a user information
55b user database
55c evacuation route database
55d family terminal information (terminal-device-related information)
55e guidance service information
55f coordinate conversion database
56 communicator (user information receiver, guidance service information transmitter)

1. An information processing system comprising a terminal device used by a user, and an information processing device configured to communicate with the terminal device,
the terminal device including
a physiological information acquisition unit that acquires physiological information of the user,
a positioning information acquisition unit that acquires positioning information identifying the position of the terminal device,
a user information transmitter that sends user information including physiological information acquired by the physiological information acquisition unit, and positioning information acquired by the positioning information acquisition unit, to the information processing device, and
a display that displays specific grid coordinates converted from latitude and longitude;
the information processing device including
a user information receiver that receives the user information transmitted by the terminal device,
a guidance service information generator that generates, based on the user information received by the user information receiver, guidance service information that is provided to the user, and
a guidance service information transmitter that sends the guidance service information the guidance service information generator generated to the terminal device.

2. The information processing system described in claim 1, wherein:
the guidance service information is information for guiding the user to a specific guidance location, and is configured so that the guidance location is changed according to the physiological information of the user.

3. The information processing system described in claim 1, wherein:
the terminal device includes a body contact unit that contacts the body of the user;
the physiological information acquisition unit detects bio-information through the body contact unit, and acquires the detected bio-information as the physiological information; and
the guidance service information generator of the information processing device generates the guidance service information based on the physiological information contained in the user information received by the user information receiver.

4. An information processing device configured to communicate with a terminal device used by a user, comprising:
a user information receiver that receives, from the terminal device, user information including positioning information identifying the position of the terminal device, and physiological information of the user;
a guidance service information generator that generates, based on the user information received by the user information receiver, guidance service information that is provided to the user; and
a guidance service information transmitter that sends the guidance service information the guidance service information generator generated to the terminal device,
the information processing device sending guidance service information including specific grid coordinates converted from latitude and longitude to the terminal device.

5. The information processing device described in claim 4, having terminal-device-related information relating the terminal device to another terminal device;
the guidance service information generator including, in the guidance service information corresponding to the terminal device, information related to another terminal device that is related to the terminal device by the terminal-device-related information.

6. The information processing device described in claim 5, wherein:
the guidance service information generator generates the guidance service information corresponding to another terminal device based on the user information received by the user information receiver from the terminal device.

7. The information processing device described in claim 4, wherein:
the guidance service information generator generates the guidance service information to include positioning information in a format defined for the information processing device.

8. The information processing device described in claim 4, wherein:
the guidance service information transmitter sends guidance service information sent to the terminal device to another predetermined destination.

9. A terminal device communicatively connected to an information processing device, comprising:
a physiological information acquisition unit that acquires physiological information of the user of the terminal device;
a positioning information acquisition unit that acquires positioning information identifying the position of the terminal device;
a user information transmitter that sends user information including physiological information acquired by the physiological information acquisition unit, and positioning information acquired by the positioning information acquisition unit, to the information processing device;
a guidance service information receiver that receives guidance service information transmitted from the information processing device corresponding to the user information sent by the user information transmitter;
a body contact unit that contacts the body of the user; and
a display that displays specific grid coordinates converted from latitude and longitude,
the terminal device being configured to be usable when the body contact unit is touching the body of the user.

10. The terminal device described in claim 9, wherein:
the physiological information acquisition unit detects bio-information through the body contact unit, and acquires the detected bio-information as the physiological information.

11. The terminal device described in claim 9, comprising:
terminal storage that stores physical characteristics related to the body of the user;
the physiological information acquisition unit acquiring the physiological information based on the physical characteristics stored by the terminal storage.

12. The terminal device described in claim 9, wherein:
the positioning information acquisition unit detects the position of the terminal device and generates positioning information identifying the detected position.

13. The terminal device described in claim 9, comprising:
a terminal display that displays guidance service information received by the guidance service information receiver;
the terminal display displaying positioning information contained in the guidance service information as positioning information in a format defined for the information processing device.

14. The terminal device described in claim 9, comprising:
a power generator that produces power, and a power supply unit that supplies at least power generated by the power generator to the user information transmitter and guidance service information receiver.

15. The terminal device described in claim 9, comprising:
a motion detector that detects movement of the terminal device;
the user information transmitter sending the detection result of the motion detector to the information processing device.

16. An information processing method using a terminal device used by a user, and an information processing device configured to communicate with the terminal device, comprising:
the terminal device
acquiring physiological information of the user,
acquiring positioning information identifying the position of the terminal device, and
sending user information including the physiological information and the positioning information to the information processing device; and
the information processing device
receiving the user information transmitted by the terminal device,
generating guidance service information including specific grid coordinates converted from latitude and longitude based on the user information, and
sending the guidance service information to the terminal device.

17. An information processing system comprising a terminal device used by a user, and an information processing device configured to communicate with the terminal device,
the terminal device including
a physiological information acquisition unit that acquires physiological information of the user,
a positioning information acquisition unit that acquires positioning information identifying the position of the terminal device,
a terminal receiver that receives a query sent by the information processing device,
a user information transmitter that, when the terminal receiver receives the query, sends user information including physiological information acquired by the physiological information acquisition unit, and positioning information acquired by the positioning information acquisition unit, to the information processing device, and
a display that displays specific grid coordinates converted from latitude and longitude; and
the information processing device including
a query transmitter that sends a query to the terminal device, and
a user information receiver that receives the user information from the terminal device.

18. The information processing system described in claim 17, wherein:
the information processing device receives the user information from multiple terminal devices, and displays disaster conditions based on the user information.

19. The information processing system described in claim 17, wherein:
the information processing device includes user information storage that stores information combining the positioning information and the physiological information contained in the user information received by the user information receiver.

20. The information processing system described in claim 17, wherein:
the physiological information acquisition unit of the terminal device includes a bio-information detector that contacts the body of the user and detects bio-information, and acquires, and transmits by the user information transmitter, the bio-information detected by the bio-information detector as the physiological information.

21. The information processing system described in claim 17, wherein:
the terminal device is connected to the information processing device through a communication network including communication devices disposed appropriately in a region; and
the query transmitter of the information processing device specifying a region for the communication devices when sending the query to the terminal devices.

22. An information processing device configured to communicate with a terminal device used by a user, comprising:
a query transmitter that sends a query to the terminal device; and
a user information receiver that receives user information that is sent by the terminal device in response to a query sent by the transmitter, and includes positioning information identifying the position of the terminal device, and physiological information of the user,
the information processing device sending guidance service information including specific grid coordinates converted from latitude and longitude to the terminal device.

23. An information processing method using a terminal device used by a user, and an information processing device capable of communicating with the terminal device, comprising:
the terminal device
acquiring physiological information of the user,
acquiring positioning information identifying the position of the terminal device,
receiving a query transmitted by the information processing device, and
when the query is received, sending user information including the physiological information and specific grid coordinates converted from latitude and longitude to the information processing device; and
the information processing device
sending a query to the terminal device, and
receiving the user information from the terminal device.

24. The terminal device that is used in the information processing system described in claim 17.
